(19) United States
(12) Patent Application Publication
(10) Pub. No.: US 2006/0166879 A1
(43) Pub. Date: Jul. 27, 2006

(54) Treatment of conditions associated with the presence of macromolecular aggregates, particularly ophthalmic disorders

(75) Inventors: Rajiv Bhushan, Palo Alto, CA (US); Jerry B. Gin, Sunnyvale, CA (US)

Correspondence Address: MINTZ, LEVIN, COHN, FERRIS, GLOVSKY AND POPEO, P.C, 1400 PAGE MILL ROAD, PALO ALTO, CA, 94304-1124, US

(73) Assignee: Chakshu Research Inc, Los Gatos, CA (US)

(21) Appl. No.: 11/182,999

(22) Filed: Jul. 15, 2005

Related U.S. Application Data
(63) Continuation-in-part of application No. 10/744,524, filed on Dec. 22, 2003.
(60) Provisional application No. 60/435,849, filed on Dec. 20, 2002.
(60) Provisional application No. 60/506,474, filed on Sep. 26, 2003.

(51) Int. Cl. A61K 38/17; A61K 48/00; A61K 31/185

(52) U.S. Cl. 514/12; 514/44; 514/553

(57) ABSTRACT

A method and formulation are provided for the treatment of medical conditions associated with the formation and/or deposition of macromolecular aggregates, particularly those associated with adverse ocular conditions. The formulation contains a non-cytotoxic chelating agent containing at least three negatively charged chelating atoms and a charge-masking agent containing at least one polar group and having a molecular weight of less than about 250, wherein the polar group contains at least one and preferably at least two heteroatoms having a Pauling electronegativity greater than about 3.00, and further wherein the molar ratio of the charge-masking agent to the chelating agent is sufficient to ensure that substantially all negatively charged chelating atoms are associated with a heteroatom on the charge-masking agent.

### CROSS-REFERENCE TO RELATED APPLICATIONS

This application is a continuation in part of U.S. patent application Ser. No. 10/744,524, filed Dec. 22, 2003, which in turn claims priority under 35 U.S.C. §119(e)(1) to provisional U.S. Patent Application Ser. No. 60/435,849, filed Dec. 20, 2002, and to provisional U.S. Patent Application Ser. No. 60/506,474, filed Sep. 26, 2003. The disclosures of these applications are incorporated by reference herein.

### TECHNICAL FIELD

This invention relates generally to the treatment of disorders, diseases, and other adverse medical conditions, including the adverse ocular conditions disorders often associated with aging. More particularly, the invention pertains to the treatment of conditions associated with the presence of macromolecular aggregates such as may be present in the eye. The invention finds utility in a variety of fields, including ophthalmology and geriatrics.

### BACKGROUND

Progressive, age-related changes of the eye, including normal as well as pathological changes, have always been an unwelcome but inevitable part of extended life in humans and other mammals. Many of these changes seriously affect both the function and the cosmetic appearance of the eyes. These changes include: development of cataracts; hardening, opacification, reduction of pliability, and yellowing of the lens; yellowing and opacification of the cornea; presbyopia; clogging of the trabeculum, leading to intraocular pressure build-up and glaucoma; increased floaters in the vitreous humor; stiffening and reduction of the dilation range of the iris; age-related macular degeneration (AMD); formation of atherosclerotic deposits in retinal arteries; dry eye syndrome; and decreased sensitivity and light level adaptation ability of the rods and cones of the retina. Age-related vision deterioration includes loss in visual acuity, visual contrast, color and depth perception, lens accommodation, light sensitivity, and dark adaptation. Age-related changes also include changes in the color appearance of the iris, and formation of arcus senilis. The invention is, in large part, directed toward a formulation and method for preventing and treating a multiplicity of age-related ocular disorders and diseases.

All parts of the eye, including the cornea, sclera, trabeculum, iris, lens, vitreous humor, and retina are affected by the aging process, as explained below.

The Cornea:

The cornea is the eye's outermost layer. It is the clear, dome-shaped surface that covers the front of the eye. The cornea is composed of five layers. The epithelium is a layer of cells that forms the surface. It is only about 5-6 cell layers thick and quickly regenerates when the cornea is injured. If an injury penetrates more deeply into the cornea, scarring may occur and leave opaque areas, causing the cornea to lose its clarity and luster. Immediately below the epithelium is Bowman's membrane, a protective layer that is very tough and difficult to penetrate. The stroma, the thickest layer of the cornea, lies just beneath Bowman's membrane and is composed of tiny collagen fibrils aligned in parallel, an arrangement that provides the cornea with its clarity. Descemet's membrane underlies the stroma and is just above the innermost corneal layer, the endothelium. The endothelium is just one cell layer in thickness, and serves to pump water from the cornea to the aqueous, keeping it clear. If damaged or diseased, these cells will not regenerate.

As the eye ages, the cornea can become more opaque. Opacification can take many forms. The most common form of opacification affects the periphery of the cornea, and is termed “arcus senilis,” or “arcus.” This type of opacification initially involves deposition of lipids into Descemet's membrane. Subsequently, lipids deposit into Bowman's membrane and possibly into the stroma as well. Arcus senilis is usually not visually significant, but is a cosmetically noticeable sign of aging. There are other age related corneal opacifications, however, which may have some visual consequences. These include central cloudy dystrophy of Francois, which affects the middle layers of the stroma, and posterior crocodile shagreen, which is central opacification of the posterior stroma. Opacification, by scattering light, results in progressive reduction of visual contrast and visual acuity.

Opacification of the cornea develops as a result of a number of factors, including, by way of example: degeneration of corneal structure; cross-linking of collagen and other proteins by metalloproteinases; ultraviolet (UV) light damage; oxidation damage; and buildup of substances like calcium salts, protein waste, and excess lipids.

There is no established treatment for slowing or reversing corneal changes other than surgical intervention. For example, opaque structures can be scraped away with a blunt instrument after first removing the epithelium, followed by smoothing and sculpting the corneal surface with a laser beam. In severe cases of corneal scarring and opacification, corneal transplantation has been the only effective approach.

Another common ocular disorder that adversely affects the cornea as well as other structures within the eye is keratoconjunctivitis sicca, commonly referred to as “dry eye syndrome” or “dry eye.” Dry eye can result from a host of causes, and is frequently a problem for older people. The disorder is associated with a scratchy sensation, excessive secretion of mucus, a burning sensation, increased sensitivity to light, and pain. Dry eye is currently treated with “artificial tears,” a commercially available product containing a lubricant such as low molecular weight polyethylene glycol. Surgical treatment, also, is not uncommon, and usually involves insertion of a punctal plug so that lacrimal secretions are retained in the eye. However, both types of treatment are problematic: surgical treatment is invasive and potentially risky, while artifical tear products provide only very temporary and often inadequate relief.

The Sclera:

The sclera is the white of the eye. In younger individuals, the sclera has a bluish tinge, but as people grow older, the sclera yellows as a result of age-related changes in the conjunctiva. Over time, UV and dust exposure may result in changes in the conjunctival tissue, leading to pingecula and pterygium formation. These ocular growths can further cause breakdown of scleral and corneal tissue. Currently, surgery, including conjunctival transplantation, is the only accepted treatment for pingeculae and pterygia.

The Trabeculum:

The trabeculum, also referred to as the trabecular meshwork, is a mesh-like structure located at the iris-sclera junction in the anterior chamber of the eye. The trabeculum serves to filter aqueous fluid and control its flow from the anterior chamber into the canal of Schlemm. As the eye ages, debris and protein-lipid waste may build up and clog the trabeculum, a problem that results in increased pressure within the eye, which in turn can lead to glaucoma and damage to the retina, optic nerve, and other structures of the eye. Glaucoma drugs can help reduce this pressure, and surgery can create an artificial opening to bypass the trabeculum and reestablish flow of liquid out of the vitreous and aqueous humor. There is, however, no known method for preventing a build-up of debris and protein-lipid waste within the trabeculum.

The Iris and Pupil:

With age, dilation and constriction of the iris in response to changes in illumination become slower, and its range of motion decreases. Also, the pupil becomes progressively smaller with age, severely restricting the amount of light entering the eye, especially under low light conditions. The narrowing pupil and the stiffening, slower adaptation, and constriction of the iris over time are largely responsible for the difficulty the aged have in seeing at night and adapting to changes in illumination. The changes in iris shape, stiffness, and adaptability are generally thought to come from fibrosis and cross-linking between structural proteins. Deposits of protein and lipid wastes on the iris over time may also lighten its coloration. Both the light-colored deposits on the iris, and narrowing of the pupil, are very noticeable cosmetic markers of age that may have social implications for individuals. There is no standard treatment for any of these changes, or for changes in iris coloration with age.

The Lens:

With age, the lens yellows, becomes harder, stiffer, and less pliable, and can opacity either diffusely or in specific locations. Thus, the lens passes less light, which reduces visual contrast and acuity. Yellowing also affects color perception. Stiffening of the lens as well as the inability of the muscle to accommodate the lens results in a condition generally known as presbyopia. Presbyopia, almost always occurring after middle age, is the inability of an eye to focus correctly. This age-related ocular pathology manifests itself in a loss of accommodative ability, i.e., the capacity of the eye, through the lens, to focus on near or far objects by changing the shape of the lens to become more spherical (or convex). Both myopic and hyperopic individuals are subject to presbyopia. The age-related loss of accommodative amplitude is progressive, and presbyopia is perhaps the most prevalent of all ocular afflictions, ultimately affecting virtually all individuals during the normal human life span.

These changes in the lens are thought to be due to degenerative changes in the structure of the lens, including glycated crosslinks between collagen fibers, buildup of protein complexes, ultraviolet light degradation of structures, oxidation damage, and deposits of waste proteins, lipids, and calcium salts. Elastic and viscous properties of the lens are dependent on properties of the fiber membranes and cytoskeleton crystallins. The lens fiber membranes are characterized by an extremely high cholesterol to phospholipid ratio. Any changes in these components affect the deformability of the lens membrane. The loss of lens deformability has also been attributed to increased binding of lens proteins to the cell membranes.

Compensatory options to alleviate presbyopia currently include bifocal reading glasses and/or contact lenses, monovision intraocular lenses (IOLs) and/or contact lenses, multifocal IOLs, monovision and anisometropic corneal refractive surgical procedures using radial keratotomy (RK), photorefractive keratomileusis (PRK), and laser-assisted in situ keratomileusis (LASIK). No universally accepted treatments or cures are currently available for presbyopia.

Opacity of the lens results in an abnormal condition generally known as cataract. Cataract is a progressive ocular disease, which subsequently leads to lower vision. Most of this ocular disease is age-related senile cataract. The incidence of cataract formation is thought to be 60-70% in persons in their sixties and nearly 100% in persons eighty years or older. However, at the present time, there is no agent that has been clearly proven to inhibit the development of cataracts. Therefore, the development of an effective therapeutic agent has been desired. Presently, the treatment of cataracts depends upon the correction of vision using eyeglasses, contact lenses, or surgical operations such as insertion of an intra-ocular lens into the capsula lentis after extra-capsular cataract extraction.

In cataract surgery, the incidence of secondary cataract after surgery has been a problem. Secondary cataract is equated with opacity present on the surface of the remaining posterior capsule following extracapsular cataract extraction. The mechanism of secondary cataract is mainly as follows. After excising lens epithelial cells (anterior capsule), secondary cataract results from migration and proliferation of residual lens epithelial cells, which are not completely removed at the time of extraction of the lens cortex, onto the posterior capsule leading to posterior capsule opacification. In cataract surgery, it is impossible to remove lens epithelial cells completely, and consequently it is difficult to always prevent secondary cataract. It is said that the incidence of the above posterior capsule opacification is 40-50% in eyes that do not receive an intracapsular posterior chamber lens implant and 7-20% in eyes which do receive an intracapsular lens implant. Additionally, eye infections categorized as endophthalmitis have also been observed after cataract surgeries.

The Vitreous Humor:

Floaters are debris particles that interfere with clear vision by projecting shadows on the retina. There currently is no standard treatment for reducing or eliminating floaters.

The Retina:

A number of changes can occur in the retina with age. Atherosclerotic buildup and leakage in the retinal arteries can lead to macular degeneration as well as reduction of peripheral vision. The rods and cones can become less sensitive over time as they replenish their pigments more slowly. Progressively, all these effects can reduce vision, ultimately leading to partial or complete blindness. Retinal diseases such as age-related macular degeneration have been hard to cure. Current retinal treatments include laser surgery to stop the leaking of blood vessels in the eye.

As alluded to above, current therapeutic attempts to address many ocular disorders and diseases, including aging-related ocular problems, often involve surgical intervention. Surgical procedures are, of course, invasive, and, furthermore, often do not achieve the desired therapeutic goal. Additionally, surgery can be very expensive and may result in significant undesired after-effects. For example, secondary cataracts may develop after cataract surgery and infections may set in. Endophthalmitis has also been observed after cataract surgery. In addition, advanced surgical techniques are not universally available, because they require a very well developed medical infrastructure. Therefore, it would be of significant advantage to provide straightforward and effective pharmacological therapies that obviate the need for surgery.

There have been products proposed to address specific, individual aging-related ocular conditions. For example, artificial tears and herbal formulations such as Simalasan eyedrops have been suggested for treating dry eye syndrome, and other eyedrops are available to reduce intraocular pressure, alleviate discomfort, promote healing after injury, reduce inflammation, and prevent infections. However, self-administration of multiple products several times a day is inconvenient, potentially results in poor patient compliance (in turn reducing overall efficacy), and can involve detrimental interaction of formulation components. For example, the common preservative benzalkonium chloride may react with other desirable components such as ethylenediamine tetraacetic acid (EDTA). Accordingly, there is a need in the art for a comprehensive pharmaceutical formulation that can prevent, arrest, and/or reverse a multiplicity of aging-related vision problems and the associated ocular disorders.

To date, such a formulation has not been provided, in large part because complex, multi-component pharmaceutical products are often problematic for formulators and manufacturers. Problems can arise, for example, from combining agents having different solubility profiles and/or membrane transport rates. With respect to the latter consideration, transport facilitators, also termed “permeation enhancers,” need to be incorporated into a formulation, and must be pharmaceutically acceptable, have no effect on formulation stability, and be inert to and compatible with other components of the formulation and the physiological structures with which the formulation will come into contact.

Many adverse ocular conditions are associated with the formation, presence, and/or growth of macromolecular aggregates in the eye. Indeed, many pathologies result from or are associated with the deposition and/or aggregation of proteins, other peptidyl species, lipoproteins, lipids, polynucleotides, and other macromolecules throughout the body. For example, Advanced Glycation Endproducts (also termed AGEs) are formed by the binding of glucose or other reducing sugars to proteins, lipoproteins and DNA by a process known as non-enzymatic glycation, followed by cross-linking. These cross-linked macromolecules stiffen connective tissue and lead to tissue damage in the kidney, retina, vascular wall and nerves. AGEs have, in fact, been implicated in the pathogenesis of a variety of debilitating diseases such as diabetes, atherosclerosis, Alzheimer's and rheumatoid arthritis, as well as in the normal aging process. Peptidyl deposits are also associated with Alzheimer's disease, sickle cell anemia, multiple myeloma, and prion diseases. Lipids, particularly sterols and sterol esters, represent an additional class of biomolecules that form pathogenic deposits in vivo, including atherosclerotic plaque, gallstones, and the like. To date, there has been no single formulation identified capable of treating a plurality of such disorders.

### SUMMARY OF THE INVENTION

The present invention is directed to the aforementioned need in the art, and, in one embodiment, provides a method for eliminating or reducing the size of an aggregate of macromolecules in the eye, the method comprising administering a therapeutically effective amount of an ophthalmic formulation comprised of (a) a noncytotoxic chelating agent containing at least three negatively charged chelating atoms, and (b) a charge-masking agent containing at least one polar group and having a molecular weight less than about 250. The polar group contains at least one and preferably at least two heteroatoms having a Pauling electronegativity greater than about 3.00, wherein the heteroatoms are preferably oxygen atoms. The molar ratio of the charge-masking agent to the chelating agent is sufficient to ensure that substantially all negatively charged chelating atoms are associated with one of the aforementioned heteroatoms on the charge-masking agent.

As there are many ocular disorders associated with the formation or deposition of macromolecular aggregates, it will be appreciated that the invention has utility in the prevention and treatment of a host of adverse ocular conditions, including Age-Related Macular Degeneration (AMD), diabetic retinopathy, and glaucoma. The invention also pertains to methods of using the formulation in the prevention and treatment of adverse ocular conditions that involve oxidative and/or free radical damage in the eye, some of which are also associated with the formation or deposition of macromolecular aggregates. These adverse ocular conditions include, by way of example, conditions, diseases, or disorders of the cornea, retina, lens, sclera, and anterior and posterior segments of the eye. An adverse ocular condition as that term is used herein may be a “normal” condition that is frequently seen in aging individuals (e.g., decreased visual acuity and contrast sensitivity) or a pathologic condition that may or may not be associated with the aging process. The latter adverse ocular conditions include a wide variety of ocular disorders and diseases. Aging-related ocular problems that can be prevented and/or treated using the present formulations include, without limitation, opacification (both corneal and lens opacification), cataract formation (including secondary cataract formation) and other problems associated with deposition of lipids, visual acuity impairment, decreased contrast sensitivity, photophobia, glare, dry eye, loss of night vision, narrowing of the pupil, presbyopia, age-related macular degeneration, elevated intraocular pressure, glaucoma, and arcus senilis. By “aging-related” is meant a condition that is generally recognized as occurring far more frequently in older patients, but that may and occasionally do occur in younger people. The formulations can also be used in the treatment of ocular surface growths such as pingueculae and pterygia, which are typically caused by dust, wind, or ultraviolet light, but may also be symptoms of degenerative diseases associated with the aging eye. Another adverse condition that is generally not viewed as aging-related but which can be treated using the present formulation includes keratoconus. It should also be emphasized that the present formulation can be advantageously employed to improve visual acuity, in general, in any mammalian individual. That is, ocular administration of the formulation can improve visual acuity and contrast sensitivity as well as color and depth perception regardless of the patient's age or the presence of any adverse ocular conditions.

In a further embodiment, the invention provides a method, formulation, and implant for the prevention or treatment of cataracts, including secondary cataracts. The method involves ocular administration of a formulation as defined above, i.e., a formulation comprised of (a) a noncytotoxic chelating agent containing at least three negatively charged chelating atoms, and (b) a charge-masking agent containing at least one polar group and having a molecular weight less than about 250, wherein the polar group contains at least one and preferably at least two heteroatoms having a Pauling electronegativity greater than about 3.00, and further wherein the molar ratio of the charge-masking agent to the chelating agent is sufficient to ensure that substantially all negatively charged chelating atoms are associated with at least one of the aforementioned heteroatoms on the charge-masking agent.

In another embodiment, a pharmaceutical formulation is provided that comprises:

(a) a noncytotoxic chelating agent containing at least three negatively charged chelating atoms;

(b) a charge-masking agent containing at least one polar group and having a molecular weight less than about 250, wherein the polar group contains at least one and preferably at least two heteroatoms having a Pauling electronegativity greater than about 3.00, and further wherein the molar ratio of the charge-masking agent to the chelating agent is sufficient to ensure that substantially all negatively charged chelating atoms are associated with a heteroatom in a polar group on the charge-masking agent; and

(c) a pharmaceutically acceptable aqueous vehicle.

The ophthalmic formulation may be administered in any form suitable for ocular drug administration, e.g., as a solution, suspension, ointment, gel, liposomal dispersion, colloidal microparticle suspension, or the like, or in an ocular insert, e.g., in an optionally biodegradable controlled release polymeric matrix. Significantly, at least one component of the formulation, and preferably two or more formulation components, are “multifunctional” in that they are useful in preventing or treating multiple conditions and disorders, or have more than one mechanism of action, or both. Accordingly, the present formulations eliminate a significant problem in the art, namely, cross-reaction between different formulation types and/or active agents when multiple formulations are used to treat a patient with multiple ocular disorders. Additionally, in a preferred embodiment, the formulation is entirely composed of components that are naturally occurring and/or as GRAS (“Generally Regarded as Safe”) by the U.S. Food and Drug Administration.

The invention also pertains to ocular inserts for the controlled release of a chelating agent as noted above, e.g., EDTA, and/or a charge-masking agent such as methylsulfonylmethane. The insert may be a gradually but completely soluble implant, such as may be made by incorporating swellable, hydrogel-forming polymers into an aqueous liquid formulation. The insert may also be insoluble, in which case the agent or agents are released from an internal reservoir through an outer membrane via diffusion or osmosis.

### BRIEF DESCRIPTION OF THE FIGURES

The patent or application file contains at least one drawing executed in color. Copies of this patent or patent application publication with color drawings will be provided by the Office upon request and payment of the necessary fee.

FIGS. 1A, 1B, 2A, and 2B are photographs of the eyes of a 46-year-old male subject prior to treatment (OD-FIG. 1A; OS-FIG. 2A) and after (OS-FIG. 1B; and OS-FIG. 2B) receiving eight weeks of treatment with an eye drop formulation of the invention, as described in Example 5.

FIGS. 3A, 3B, 4A, and 4B are photographs of the eyes of a 60-year-old male subject prior to treatment (OD-FIG. 3A; OS-FIG. 4A) and after (OS-FIG. 3B; and OS-FIG. 3B) receiving eight weeks of treatment with an eye drop formulation of the invention, as described in Example 6.

FIG. 5 compares the contrast sensitivity improvement resulting from Formulation 3 compared to placebo in Example 14.

FIG. 6 compares the penetration of solutions A, B, and C in Example 15 after 30 minutes, 2 hours, and 16 hours.

FIGS. 7A and 7B depict the permeation of EDTA as found in Example 16.

FIGS. 8A and 8B depict the effect of various treatments from Example 17.

FIG. 9 depicts the transmission in rat lenses as a function of treatment in Example 17.

FIG. 10 depicts the effect of various treatments on cell viability as found in Example 18.

### DETAILED DESCRIPTION OF EMBODIMENTS OF THE INVENTION

Unless otherwise indicated, the invention is not limited to specific formulation types, formulation components, dosage regimens, or the like, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a chelating agent” includes a single such agent as well as a combination or mixture of two or more different chelating agents, reference to “a charge-masking agent” includes not only a single charge-masking agent but also a combination or mixture of two or more different charge-masking agents, reference to “a pharmaceutically acceptable vehicle” includes two or more such vehicles as well as a single vehicle, and the like.

In this specification and in the claims that follow, reference will be made to a number of terms, which shall be defined to have the following meanings:

When referring to a formulation component, it is intended that the term used, e.g., “agent” or “component,” encompass not only the specified molecular entity but also its pharmaceutically acceptable analogs, including, but not limited to, salts, esters, amides, prodrugs, conjugates, active metabolites, and other such derivatives, analogs, and related compounds.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible to a particular adverse condition, disorder, or disease, and thus relates to the prevention of the occurrence of symptoms and/or their underlying cause. Unless otherwise indicated herein, either explicitly or by implication, if the term “treatment” (or “treating”) is used without reference to possible prevention, it is intended that prevention be encompassed as well, such that “a method for the treatment of presbyopia” would be interpreted as encompassing “a method for the prevention of presbyopia.”

By the terms “effective amount” and “therapeutically effective amount” of a formulation or formulation component is meant a nontoxic but sufficient amount of the formulation or component to provide the desired effect.

The term “controlled release” refers to an agent-containing formulation or fraction thereof in which release of the agent is not immediate, i.e., with a “controlled release” formulation, administration does not result in immediate release of the agent into an absorption pool. The term is used interchangeably with “nonimmediate release” as defined in Remington: The Science and Practice of Pharmacy, Nineteenth Ed. (Easton, Pa.: Mack Publishing Company, 1995). In general, the term “controlled release” as used herein refers to “sustained release” rather than to “delayed release” formulations. The term “sustained release” (synonymous with “extended release”) is used in its conventional sense to refer to a formulation that provides for gradual release of an agent over an extended period of time.

By a “pharmaceutically acceptable” or “ophthalmologically acceptable” component is meant a component that is not biologically or otherwise undesirable, i.e., the component may be incorporated into an ophthalmic formulation of the invention and administered topically to a patient's eye without causing any undesirable biological effects or interacting in a deleterious manner with any of the other components of the formulation composition in which it is contained. When the term “pharmaceutically acceptable” is used to refer to a component other than a pharmacologically active agent, it is implied that the component has met the required standards of toxicological and manufacturing testing or that it is included on the Inactive Ingredient Guide prepared by the U.S. Food and Drug Administration.

The phrase “having the formula” or “having the structure” is not intended to be limiting and is used in the same way that the term “comprising” is commonly used.

The term “alkyl” as used herein refers to a linear, branched, or cyclic saturated hydrocarbon group containing 1 to 6 carbon atoms, such as methyl, ethyl, n-propyl, isopropyl, n-butyl, isobutyl, t-butyl, cyclopentyl, cyclohexyl and the like. If not otherwise indicated, the term “alkyl” includes unsubstituted and substituted alkyl, wherein the substituents may be, for example, halo, hydroxyl, sulfhydryl, alkoxy, acyl, etc.

The term “alkoxy” as used herein intends an alkyl group bound through a single, terminal ether linkage; that is, an “alkoxy” group may be represented as —O-alkyl where alkyl is as defined above.

The term “aryl,” as used herein and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 14 carbon atoms. Exemplary aryl groups are contain one aromatic ring or two fused or linked aromatic rings, e.g., phenyl, naphthyl, biphenyl, diphenylether, diphenylamine, benzophenone, and the like. If not otherwise indicated, the term “aryl” includes unsubstituted and substituted aryl, wherein the substituents may be as set forth above with respect to optionally substituted “alkyl” groups.

The term “aralkyl” refers to an alkyl group with an aryl substituent, wherein “aryl” and “alkyl” are as defined above. Preferred aralkyl groups contain 6 to 14 carbon atoms, and particularly preferred aralkyl groups contain 6 to 8 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like.

The term “acyl” refers to substituents having the formula —(CO)-alkyl, —(CO)-aryl, or —(CO)-aralkyl, wherein “alkyl,” “aryl, and “aralkyl” are as defined above.

The terms “heteroalkyl” and “heteroaralkyl” are used to refer to heteroatom-containing alkyl and aralkyl groups, respectively, i.e., alkyl and aralkyl groups in which one or more carbon atoms is replaced with an atom other than carbon, e.g., nitrogen, oxygen, sulfur, phosphorus or silicon, typically nitrogen, oxygen or sulfur.

The terms “peptide” and “peptidyl” are intended to include any structure comprised of two or more amino acids. The amino acids forming all or a part of a peptide may be any of the twenty conventional, naturally occurring amino acids, i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y). Any of the amino acids may be replaced by a non-conventional amino acid such as, for example, an isomer or analog of a conventional amino acid (e.g., a D-amino acid), a non-protein amino acid, a post-translationally modified amino acid, an enzymatically modified amino acid, or a construct or structure designed to mimic an amino acid. Peptidyl compounds herein include proteins, oligopeptides, polypeptides, lipoproteins, glycosylated peptides, glycoproteins, and the like.

In one embodiment, then, a method is provided for eliminating or reducing the size of an aggregate of macromolecules in the eye. The method involves administering to the eye(s) of a patient a therapeutically effective amount of a sterile ophthalmic formulation comprised of (a) a noncytotoxic chelating agent containing at least three negatively charged chelating atoms, and (b) a charge-masking agent containing at least one polar group and having a molecular weight less than about 250. The polar group contains at least one and preferably at least two heteroatoms having a Pauling electronegativity greater than about 3.00, wherein the heteroatoms are preferably oxygen atoms. The molar ratio of the charge-masking agent to the chelating agent is sufficient to ensure that substantially all negatively charged chelating atoms are associated with at least one of the aforementioned heteroatoms on the charge-masking agent. The formulation may be applied to the eye in any form suitable for ocular drug administration, e.g., as a solution or suspension for administration as eye drops or eye washes, as an ointment, or in an ocular insert that can be implanted in the conjunctiva, sclera, pars plana, anterior segment, or posterior segment of the eye. Such inserts provide for controlled release of the formulation to the ocular surface, typically sustained release over an extended time period.

The formulation may also be applied to the skin around the eye for penetration therethrough, insofar as the compound used as the charge-masking agent, e.g., methylsulfonylmethane, also serves as a penetration enhancer allowing permeation of the formulation through the skin.

Compounds useful as chelating agents herein include any compounds that coordinate to or form complexes with a divalent or polyvalent metal cation, thus serving as a sequestrant of such cations. Accordingly, the term “chelating agent” herein includes not only divalent and polyvalent ligands (which are typically referred to as “chelators”) but also monovalent ligands capable of coordinating to or forming complexes with the metal cation. Preferred chelating agents herein, however, are basic addition salts of a polyacid, e.g., a polycarboxylic acid, a polysulfonic acid, or a polyphosphonic acid, with polycarboxylates particularly preferred. The chelating agent generally represents about 0.6 wt. % to 10 wt. %, preferably about 1.0 wt. % to 5.0 wt. %, of the formulation.

Suitable biocompatible chelating agents useful in conjunction with the present invention include, without limitation, monomeric polyacids such as EDTA, cyclohexanediamine tetraacetic acid (CDTA), hydroxyethylethylenediamine triacetic acid (HEDTA), diethylenetriamine pentaacetic acid (DTPA), dimercaptopropane sulfonic acid (DMPS), dimercaptosuccinic acid (DMSA), aminotrimethylene phosphonic acid (ATPA), citric acid, pharmaceutically acceptable salts thereof, and combinations of any of the foregoing. Other exemplary chelating agents include: phosphates, e.g., pyrophosphates, tripolyphosphates, and hexametaphosphates.

EDTA and ophthalmologically acceptable EDTA salts are particularly preferred, wherein representative ophthalmologically acceptable EDTA salts are typically selected from diammonium EDTA, disodium EDTA, dipotassium EDTA, triammonium EDTA, trisodium EDTA, tripotassium EDTA, and calcium disodium EDTA.

The following table indicates some of the common chelating agents useful in conjunction with the present invention, along with some of the cations with which they form complexes:

| | REPRESENTATIVE |
|---|---|
| | IONS |
| CHELATING AGENT | COMPLEXED |
| Bicinchoninic acid | Cu²⁺, Cu⁺ |
| Calcein (Fluorescein-methylene- | Ca²⁺, Mg²⁺ |
| iminodiacetic acid) | |
| Tiron (4,5-Dihydroxy-m-benzenedisulfonic | Al³⁺ |
| acid) | |
| Alizarin Red S (3,4-dihydroxy-2-anthra- | Ca²⁺ |
| quinonesulfonic acid) | |
| EDTA (ethylenediamine tetraacetic acid) | Fe²⁺, most divalent cations |
| CDTA (cyclodiamine tetracetic acid) | Fe²⁺, most divalent cations |
| EGTA (ethylene glycol bis (β- | Fe²⁺, most divalent cations |
| aminoethylether)-N,N,N′,N′- | |
| tetraacetic acid) | |
| HEDTA (hydroxyethylethylenediamine | Fe²⁺, most divalent cations |
| triacetic acid) | |
| DPTA (diethylenetriamine pentaacetic acid) | Fe²⁺, most divalent cations |
| DMPS (dimercaptopropane sulfonic acid) | Fe²⁺, most divalent cations |
| DMSA (dimercaptosuccinic acid) | Fe²⁺, most divalent cations |
| ATPA (aminotrimethylene phosphonic acid) | Fe²⁺, most divalent cations |
| CHX-DTPA (Cyclohexyl | Fe²⁺, most divalent cations |
| diethylenetriamino-pentaacetate) | |
| Citric acid | Fe²⁺ |
| 1,2-bis-(2-amino-5-fluorophenoxy)ethane- | Ca²⁺, K⁺ |
| N,N,N′,N′-tetra-acetic acid (5F-BAPTA) | |
| Arsonic acids | Zr⁴⁺, Ti⁴⁺ |
| Mandelic acid | Zr⁴⁺, Hf⁴⁺ |
| Anthranilic acid | Ni²⁺, Pb²⁺, Co, Ni²⁺, |
| | Cu²⁺, Zn^{2+,} Cd, Hg²⁺, |
| | Ag⁺ |
| 2-Furoic acid | Th⁴⁺ |
| Isooctylthioglycolic acid | Al³⁺, Fe²⁺, Cu²⁺, Bi³⁺, |
| | Sn⁴⁺, Pb²⁺, Ag⁺, Hg²⁺ |

The listing of cations in this table should not be taken to be exclusive. Many of these agents will complex to some extent with any metal cation.

The formulation also includes a a charge-masking agent containing at least one polar group and having a molecular weight less than about 250, preferably less than about 125, wherein the polar group contains at least two heteroatoms having a Pauling electronegativity greater than about 3.00, preferably oxygen atoms. The charge-masking agent will generally have the structure of formula (I) 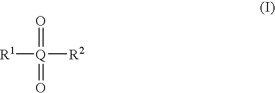
wherein the polar group is represented by the central -Q(O)₂-moiety, Q is S or P, and R¹ and R² are independently selected from C₁-C₆ alkyl (preferably C₁-C₃ alkyl), C₁-C₆ heteroalkyl (preferably C₁-C₃ heteroalkyl), C₆-C₁₄ aralkyl (preferably C₆-C₈ aralkyl), and C₂-C₁₂ heteroaralkyl (preferably C₄-C₁₀ heteraralkyl). Optimally, Q is S, and R¹ and R² are both C₁-C₃ alkyl, e.g., methyl, as in methylsulfonylmethane.

In a representative embodiment of the invention, the formulation comprises a chelating agent in the form of a basic addition salt of a tetracarboxylic acid, a charge-masking agent having the structure of formula (I) wherein R¹ and R² are independently selected from C₁-C₃ alkyl, C₁-C₃ heteroalkyl, C₆-C₈ aralkyl, and C₄-C₁₀ heteroaralkyl, and Q is S or P, and the molar ratio of the charge-masking agent to the chelating agent is in the range of 2:1 to 12:1, preferably in the range of 4:1 to 10:1,and optimally about 8:1.

The formulation can also include additional agents, e.g., a known anti-AGE agent such as an AGE breaker. As is recognized in the art, AGE breakers act to cleave glycated bonds and thus facilitate dissociation of already-formed AGEs. Suitable AGE breakers include, without limitation, L-carnosine, 3-phenacyl-4,5-dimethylthiazolium chloride (PTC), N-phenacylthiazolium bromide (PTB), and 3-phenacyl-4,5-dimethylthiazolium bromide (ALT-711, Alteon). The anti-AGE agent may also be selected from glycation inhibitors and AGE formation inhibitors. Representative such agents include aminoguanidine, 4-(2,4,6-trichlorophenylureido)phenoxyisobutyric acid, 4-[(3,4-dichlorophenylmethyl)2-chloro-phenylureido]phenoxyisobutyric acid, N,N′-bis(2-chloro-4-carboxyphenyl)formamidine, and combinations thereof.

One representative anti-AGE agent herein is L-carnosine, a natural histidine-containing dipeptide. L-carnosine is also a naturally occurring anti-oxidant, and thus provides multiple functions herein. In a preferred embodiment, L-carnosine, if present, represents approximately 0.2 wt. % to 5.0 wt. % of the formulation.

The formulation can also include a microcirculatory enhancer, i.e., an agent that serves to enhance blood flow within the capillaries. The microcirculatory enhancer can be a phosphodiesterase (PDE) inhibitor, for instance a Type (I) PDE inhibitors. Such compounds, as will be appreciated by those of ordinary skill in the art, act to elevate intracellular levels of cyclic AMP (cAMP). A preferred microcirculatory enhancer is vinpocetine, also referred to as ethyl apovincamin-22-oate. Vinpocetine, a synthetic derivative of vincamine, a Vinca alkaloid, is particularly preferred herein because of its antioxidant properties and protection against excess calcium accumulation in cells. Vincamine is also useful as a microcirculatory enhancer herein, as are Vinca alkaloids other than vinpocetine. Preferably, any microcirculatory enhancer present, e.g., vinpocetine, represents about 0.01 wt. % to about 0.2 wt. %, preferably in the range of about 0.02 wt. % to about 0.1 wt. % of the formulation.

Other optional additives in the present formulations include secondary enhancers, i.e., one or more additional permeation enhancers. For example, formulation of the invention can contain added dimethylsulfoxide (DMSO). If DMSO is added as a secondary enhancer, the amount is preferably in the range of about 1.0 wt. % to 2.0 wt. % of the formulation, and the weight ratio of MSM to DMSO is typically in the range of about 1:1 to about 50:1.

Other possible additives for incorporation into the formulations that are at least partially aqueous include, without limitation, thickeners, isotonic agents, buffering agents, and preservatives, providing that any such excipients do not interact in an adverse manner with any of the formulation's other components. It should also be noted that preservatives are not generally necessarily in light of the fact that the selected chelating agent (and preferred AGE breakers) themselves serve as preservatives. Suitable thickeners will be known to those of ordinary skill in the art of ophthalmic formulation, and include, by way of example, cellulosic polymers such as methylcellulose (MC), hydroxyethylcellulose (HEC), hydroxypropylcellulose (HPC), hydroxypropyl-methylcellulose (HPMC), and sodium carboxymethylcellulose (NaCMC), and other swellable hydrophilic polymers such as polyvinyl alcohol (PVA), hyaluronic acid or a salt thereof (e.g., sodium hyaluronate), and crosslinked acrylic acid polymers commonly referred to as “carbomers” (and available from B.F. Goodrich as Carbopol® polymers). The preferred amount of any thickener is such that a viscosity in the range of about 15 cps to 25 cps is provided, as a solution having a viscosity in the aforementioned range is generally considered optimal for both comfort and retention of the formulation in the eye. Any suitable isotonic agents and buffering agents commonly used in ophthalmic formulations may be used, providing that the osmotic pressure of the solution does not deviate from that of lachrymal fluid by more than 2-3% and that the pH of the formulation is maintained in the range of about 6.5 to about 8.0, preferably in the range of about 6.8 to about 7.8, and optimally at a pH of about 7.4. Preferred buffering agents include carbonates such as sodium and potassium bicarbonate.

The formulations of the invention also include a pharmaceutically acceptable ophthalmic carrier or vehicle, which will depend on the particular type of formulation. For example, the formulations of the invention can be provided as an ophthalmic solution or suspension, in which case the carrier is at least partially aqueous. Ideally, ophthalmic solutions, which may be administered as eye drops, are aqueous solutions. The formulations may also be ointments, in which case the pharmaceutically acceptable carrier is composed of an ointment base. Preferred ointment bases herein have a melting or softening point close to body temperature, and any ointment bases commonly used in ophthalmic preparations may be advantageously employed. Common ointment bases include petrolatum and mixtures of petrolatum and mineral oil.

In a further embodiment, the formulation can include an additional ophthalmologically active agent, such as may be selected from, for instance: anti-infective or antibiotic agents including fluoroquinolones such as ciprofloxacin, levofloxacin, gentafloxacin, ofloxacine, tetracycline, chlortetracycline, bacitracin, neomycin, polymyxin, gramicidin, oxytetracycline, chloramphenicol, gentamycin, and erythromycin; anti-inflammatory agents such as hydrocortisone, dexamethasone, fluocinolone, prednisone, prednisolone, methylprednisolone, fluorometholone, betamethasone and triamcinolone; anti-angiogenesis drugs including thalidomide, VEGF inhibitors, and matrix metaloproteinaise (MMP) inhibitors; anti-neoplastic agents; and dry-eye medicaments such as cyclosporine and mitomycin. Additonal examples of ophthalmologically active agents that may be incorporated into the present formulations include anesthetics, analgesics, cell transport/mobility impeding agents; anti-glaucoma drugs including beta-blockers such as timolol, betaxolol, atenolol, etc; carbonic anhydrase inhibitors such as acetazolamide, methazolamide, dichlorphenamide, and diamox; neuroprotectants such as nimodipine and related compounds; antibacterials such as sulfonamides, sulfacetamide, sulfamethizole and sulfisoxazole; anti-fungal agents such as fluconazole, nitrofurazone, amphotericine B, ketoconazole, and related compounds; anti-viral agents such as trifluorothymidine, acyclovir, ganciclovir, dideoxyinosine (DDI), zidovudine (AZT), foscamet, vidarabine, trifluorouridine, idoxuridine, and ribavirin; protease inhibitors and anti-cytomegalovirus agents; antiallergenics such as methapyriline, chlorpheniramine, pyrilamine and prophenpyridamine; and decongestants such as phenylephrine, naphazoline, and tetrahydrazoline.

Typical ophthalmologically active agents that can be incorporated into the present formulations include, without limitation, aceclidine, acetazolamide, anecortave, apraclonidine, atropine, azapentacene, azelastine, bacitracin, befunolol, betamethasone, betaxolol, bimatoprost, brimonidine, brinzolamide, carbachol, carteolol, celecoxib, chloramphenicol, chlortetracycline, ciprofloxacin, cromoglycate, cromolyn, cyclopentolate, cyclosporin, dapiprazole, demecarium, dexamethasone, diclofenac, dichlorphenamide, dipivefrin, dorzolamide, echothiophate, emedastine, epinastine, epinephrine, erythromycin, ethoxzolamide, eucatropine, fludrocortisone, fluorometholone, flurbiprofen, fomivirsen, framycetin, ganciclovir, gatifloxacin, gentamycin, homatropine, hydrocortisone, idoxuridine, indomethacin, isoflurophate, ketorolac, ketotifen, latanoprost, levobetaxolol, levobunolol, levocabastine, levofloxacin, lodoxamide, loteprednol, medrysone, methazolamide, metipranolol, moxifloxacin, naphazoline, natamycin, nedocromil, neomycin, norfloxacin, ofloxacin, olopatadine, oxymetazoline, pemirolast, pegaptanib, phenylephrine, physostigmine, pilocarpine, pindolol, pirenoxine, polymyxin B, prednisolone, proparacaine, ranibizumab, rimexolone, scopolamine, sezolamide, squalamine, sulfacetamide, suprofen, tetracaine, tetracyclin, tetrahydrozoline, tetryzoline, timolol, tobramycin, travoprost, triamcinulone, trifluoromethazolamide, trifluridine, trimethoprim, tropicamide, unoprostone, vidarbine, xylometazoline, a pharmaceutically acceptable salt thereof, or a combination of any of the foregoing.

The formulations of the invention may also be prepared as a hydrogel, dispersion, or colloidal suspension. Hydrogels are formed by incorporation of a swellable, gel-forming polymer such as those set forth above as suitable thickening agents (i.e., MC, HEC, HPC, HPMC, NaCMC, PVA, or hyaluronic acid or a salt thereof, e.g., sodium hyaluronate), except that a formulation referred to in the art as a “hydrogel” typically has a higher viscosity than a formulation referred to as a “thickened” solution or suspension. In contrast to such preformed hydrogels, a formulation may also be prepared so as to form a hydrogel in situ following application to the eye. Such gels are liquid at room temperature but gel at higher temperatures (and thus termed “thermoreversible” hydrogels), such as when placed in contact with body fluids. Biocompatible polymers that impart this property include acrylic acid polymers and copolymers, N-isopropylacrylamide derivatives, and ABA block copolymers of ethylene oxide and propylene oxide (conventionally referred to as “poloxamers” and available under the Pluronic® tradename from BASF-Wyandotte). The formulations can also be prepared in the form of a dispersion or colloidal suspension. Preferred dispersions are liposomal, in which case the formulation is enclosed within “liposomes,” microscopic vesicles composed of alternating aqueous compartments and lipid bilayers. Colloidal suspensions are generally formed from microparticles, i.e., from microspheres, nanospheres, microcapsules, or nanocapsules, wherein microspheres and nanospheres are generally monolithic particles of a polymer matrix in which the formulation is trapped, adsorbed, or otherwise contained, while with microcapsules and nanocapsules, the formulation is actually encapsulated. The upper limit for the size for these microparticles is about 5 μm to about 10 μm.

The formulations may also be incorporated into a sterile ocular insert that provides for controlled release of the formulation over an extended time period, generally in the range of about 12 hours to 60 days, and possibly up to 12 months or more, following implantation of the insert into the conjunctiva, sclera, or pars plana, or into the anterior segment or posterior segment of the eye. One type of ocular insert is an implant in the form of a monolithic polymer matrix that gradually releases the formulation to the eye through diffusion and/or matrix degradation. With such an insert, it is preferred that the polymer be completely soluble and or biodegradable (i.e., physically or enzymatically eroded in the eye) so that removal of the insert is unnecessary. These types of inserts are well known in the art, and are typically composed of a water-swellable, gel-forming polymer such as collagen, polyvinyl alcohol, or a cellulosic polymer. Another type of insert that can be used to deliver the present formulation is a diffusional implant in which the formulation is contained in a central reservoir enclosed within a permeable polymer membrane that allows for gradual diffusion of the formulation out of the implant. Osmotic inserts may also be used, i.e., implants in which the formulation is released as a result of an increase in osmotic pressure within the implant following application to the eye and subsequent absorption of lachrymal fluid.

The methods and formulations of the invention are useful in treating a wide variety of conditions associated with the formation and/or deposition of macromolecular aggregates. Numerous medical pathologies are caused or exacerbated by the in vivo formation or deposition of macromolecular aggregates, including crystalline aggregates, fibrillar aggregates, and amorphous aggregates. Certain peptidyl compounds, including selected oligopeptides, polypeptides, and proteins, are known to form crystals and fibrils that are associated with various medical conditions, disorders, and diseases. For example, amyloid peptides, particularly β-amyloid, are known to form ordered fibrillar aggregates that comprise the extracellular and cerebrovascular senile plaques associated with Alzheimer's disease. See Han et al. (1995), “The Core Alzheimer's Peptide NAC Forms Amyloid Fibrils which Seed and are Seeded by β-Amyloid: is NAC a Common Trigger or Target in Neurodegenerative Disease?” Chemistry and Biology 2:163-169; Serpell et al. (2000), “Molecular Structure of a Fibrillar Alzheimer's Aβ,” Biochemistry 39:13269-13275; Jarrett and Lansbury (1992), “Amyloid Fibril Formation Requires a Chemically Discriminating Nucleation Event: Studies of an Amyloidogenic Sequence from the Bacterial Protein OsmB,” Biochemistry 31(49):12345-12352; and Jarrett et al. (1993), “The Carboxy Terminus of the Beta Amyloid Protein is Critical for the Seeding of Amyloid Formation: Implications for the Pathogenesis of Alzheimer's Disease,” Biochemistry 32:4693-4697. The prion diseases, e.g., the class of diseases known as the transmissible spongiform encephalopathies, are also characterized by abnormal protein deposition in brain tissue, in which the deposits are comprised of fibrillar amyloid plaques formed primarily from the prion protein (PrP). Such diseases include scrapie transmissible mink encephalopathy, chronic wasting disease of mule deer and elk, feline spongiform encephalopathy, and bovine spongiform encephalopathy (“mad cow disease”) in animals, and Kuru, Creutzfeldt-Jakob disease, Gerstmann-Struessler-Scheinker disease, and fatal familial insomnia in humans. It has been proposed that a 15-mer amino acid sequence, PrP96-111, is responsible for initiating prion formation in vivo by providing a seed for amyloid fiber formation. See Come et al. (1993), “A Kinetic Model for Amyloid Formation in the Prion Diseases: Importance of Seeding,” Proc Natl Acad Sc. USA 90:5959-5963. Fibrillin, associated with Martan's disease, is another example of a protein that forms an ordered fibrillar structure that causes an adverse medical condition. Fibrillar plaques formed from various collagens are also associated with certain medical pathologies, e.g., cardiac diseases and collagenofibrotic glomerulopathy; see Rossi et al. (2001), “Connective Tissue Skeleton in the Normal Left Ventricle and in Hypertensive Left Ventricle Hypertrophy and Chronic Chagasic Monocarditis,” Med Sci Mon 7:820-832; Yasuda et al. (1999), “Collagenofibrotic Glomerulopathy: A Systemic Disease,” Am J Kidney Dis 33:123-127.

Other similarly problematic biomolecules include, without limitation: cystic fibrosis transmembrane conductance regulator (“CFTR”) protein, crystallization of which is associated with cystic fibrosis (see Berger et al. (2000), “Differences Between Cystic Fibrosis Transmembrane Conductance Regulator and HisP in the Interaction with the Adenine Ring of ATP,” J Biol Chem 275:29407-29412); phospholipases, which form Charcot-Leyden crystals associated with asthma, eosinophilic bone granuloma, eosinophilic pneumonia, and granulocytic leukemia (see Reginato and Kurnik (1989), “Calcium Oxalate and Other Crystals Associated with Kidney Diseases and Arthritis,” Semin Arthritis Rheum 18:198-224); cystine, which forms crystal deposits in bone marrow (associated with rickets and synovitis), the renal tubule and gastrointestinal tract (associated with cystinuria), and a variety of other body tissues, including the kidneys, eyes, and thyroid glands (associated with cystinosis, including the severe form of the disease, nephropathic cystinosis, or Fanconi's syndrome); and hemoglobin, hematoidin, cryoglobulins, and immunoglobulins (associated with hemarthrosis and other joint disorders, cryoglobulinemia, and multiple myeloma). See Gatter and Owen, Jr., “2. Crystal Identification and Joint Fluid Analysis,” in Gout, Hyperuricemia, and Other Crystal-Associated Arthropathies, Eds. Smyth et al. (New York: Marcel Dekker Inc., 1999), pp. 15-28; and Reginato and Kurnik, supra.

Lipids, particularly sterols and sterol esters, represent an additional class of biomolecules that form pathogenic deposits in vivo. Atherosclerotic plaque (atheroma) and cholesterol emboli are largely composed of cholesterol monohydrate and crystalline cholesteryl esters, including cholesteryl palmitate, oleate, linoleate, palmitoleate, linolenate, and myristate. See North et al. (1978), “The Dissolution of Cholesterol Monohydrate Crystals in Atherosclerotic Plaque Lipids,” Atherosclerosis 30:211-217; Burks and Engelman (1981), “Cholesteryl Myristate Conformation in Liquid Crystalline Mesophases Determined by Neutron Scattering,” Proc Natl Acad Sci USA 78:6863-6867; and Peng et al. (December 2000), “Quantification of Cholesteryl Esters in Human and Rabbit Atherosclerotic Plaques by Magic-Angle Spinning ¹³C-NMR,” Arterioscler Thromb Vasc Biol, pp. 2682-2688. Formation of gallstones is also associated with cholesterol crystallization, as gallstones commonly result from the crystallization of cholesterol monohydrate in bile. See Dowling (2000), “Review: Pathogenesis of Gallstones,” Aliment Pharmacol Ther 14 (Suppl. 2):39-46. Cholesterol crystals are associated with a host of additional medical pathologies, including rheumatoid arthritis, systemic lupus erythymatosis, anklosing spondylitis, bone cysts, bone granulomatosis (Erdheim-Chester disease), xanthomas, scleroderma, and paraproteinemia. Reginato and Falasca, “24. Calcium Oxalate and Other Miscellaneous Crystal Arthropathies,” in Gout, Hyperuricemia, and Other Crystal-Associated Arthropathies, supra. In the aforementioned reference, it was also proposed that crystalline deposits of other types of lipids, e.g., fatty acids, are pathogenic as well. See Reginato and Kurnik, supra. Cholesterol crystals are also observed in hypermature cataracts (e.g., Brooks, A. M. V. et al. (1994), “Crystalline nature of the iridescent particles in hypermature cataracts,” Br J Ophth 78:581-582; Knapp, H. C. (1937), “Spontaneous rupture of the lens capsule in hypermature cataract causing secondary glaucoma,” Am J Ophthalmol 20:820-821).

The method and formulations of the invention are also useful in treating a host of adverse ocular conditions, including conditions, diseases or disorders of the cornea, retina, lens, sclera, and anterior and posterior segments of the eye, many of which involve the formation or deposition of molecular aggregates as discussed above. Of particular interest are those adverse ocular conditions associated with the aging process and/or oxidative and free radical damage to the eye. By way of example and not limitation, the formulations are useful in treating the following adverse ocular conditions that are generally associated with aging: hardening, opacification, reduction of pliability, and yellowing of the lens; yellowing and opacification of the cornea; presbyopia; clogging of the trabeculum, leading to intraocular pressure build-up and glaucoma; increased floaters in the vitreous humor; stiffening and reduction of the dilation range of the iris; age-related macular degeneration; formation of atherosclerotic and other lipid deposits in retinal arteries; dry eye syndrome; development of cataracts, including secondary cataracts; photophobia, problems with glare and a decrease in the sensitivity and light level adaptation ability of the rods and cones of the retina; arcus senilis; narrowing of the pupil; loss in visual acuity, including decreased contrast sensitivity, color perception, and depth perception; loss of night vision; decreased lens accommodation; macular edema; macular scarring; and band keratopathy. The aging individual generally suffers from more than one of these conditions, normally necessitating the self-administration of two or more different pharmaceutical products. As the methods and formulations of the invention are useful for treating multiple conditions, no additional products are needed, and, therefore, the inconvenience and inherent risk of using multiple pharmaceutical products are eliminated. Additional adverse ocular conditions that can be treated using the present formulations include keratoconus and ocular surface growths such as pingueculae and pterygia. It should also be emphasized that the formulations can be used to improve the visual acuity, including contrast sensitivity, color perception, and depth perception, in any mammalian individual whether or not the individual is afflicted with an adverse visual condition.

The invention also pertains to ocular inserts for the controlled release of a formulation of the invention or a component thereof. These ocular inserts may be implanted into any region of the eye, including the sclera and the anterior and posterior segments. The insert may be a gradually but completely soluble implant, such as may be made by incorporating swellable, hydrogel-forming polymers into an aqueous liquid formulation as described elsewhere herein. The insert may also be insoluble, in which case the agent is released from an internal reservoir through an outer membrane via diffusion or osmosis as also described elsewhere herein.

It is to be understood that while the invention has been described in conjunction with the preferred specific embodiments thereof, the foregoing description and the examples that follow are intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

### EXAMPLE 1

An eye drop formulation of the invention, Formulation 1, was prepared as follows: High purity de-ionized (DI) water (500 ml) was filtered via a 0.2 micrometer filter. MSM (27 g), EDTA (13 g), and L-carnosine (5 g) were added to the filtered DI water, and mixed until visual transparency was achieved, indicating dissolution. The mixture was poured into 10 mL bottles each having a dropper cap. On a weight percent basis, the eye drops had the following composition:

| | |
|---|---|
| Purified de-ionized water | 91.74 wt. % |
| MSM | 4.95 wt. % |
| Di-sodium EDTA | 2.39 wt. % |
| L-Carnosine | 0.92 wt. %. |

### EXAMPLE 2

Formulation 1 was evaluated for efficacy in treating four subjects, all males between 52 and 84 years of age of mixed ethnicity. Subject 1 was in his fifties and had no visual problems or detectable abnormalities of the eye. Subjects 2 and 3 were in their fifties and had prominent arcus senilis around the cornea periphery in both eyes but no other adverse ocular conditions (arcus senilis is typically considered to be a cosmetic blemish). Subject 4 was in his eighties and was suffering from cataracts and Salzmann's nodules, and reported extreme photophobia and problems with glare. This subject was having great difficulty reading newspapers, books, and information on a computer screen, because of the glare and loss in visual clarity.

The formulation was administered to the subjects, one drop (approximately 0.04 mL) to each eye, two to four times per day for a period of over 12 months. All subjects were examined by an ophthalmologist during and after 12 months. No side effects, other than minor temporary irritation at the time of administering the formulation in the eye, were reported or observed by the subjects or the ophthalmologist. All four subjects completed the study.

All subjects noticed subjective changes 4 weeks into the study. At this stage, the changes reported by the subjects included increased brightness, improved clarity of vision, and reduced glare (particularly Subject 4).

After 8 weeks, the following changes were noted: All four subjects reported greatly improved vision with regard to clarity and contrast, and indicated that daytime colors appeared to increase in brilliance. Subject 1's eyesight improved from 20/25 (after correction) to better than 20/20 (with the same correction), and his eyes turned a deeper shade of blue. Subjects 2 and 3 exhibited a significant reduction of the arcus senilis.

For Subject 4, whose vision originally with best correction had been 20/400 in his left eye and 20/200 in his right eye and had acute photophobia and glare. The glare and photophobia were reduced, and the subject started to read books, newspapers, and information on the computer screen again. The visual acuity in his right eye improved significantly, from 20/200 (with correction) to 20/60 (pinhole) (with the same correction). In his left eye, his visual acuity improved as well, from 20/400 to 20/200 (with the same correction). In his left eye, he continued to have a central dark spot due to macular scarring.

After 16 weeks, the following changes were noted: All subjects reported continuing improvement of vision, including night vision, as well as improved contrast sensitivity and continued improvement in color perception. Subject 1's eyesight continued to improve, from 20/20 (after correction) to 20/15 (with the same correction). Subjects 2 and 3 continued to exhibit a reduction of the arcus senilis.

Subject 4 reported a further reduction in glare and photophobia, and further improvements in the ease of reading books, newspapers, and information on the computer screen. Subject 4 also reported that nighttime glare had been eliminated. The subject was now comfortable in daylight without need for dark glasses, and without suffering severe problems with glare. The visual acuity in his right eye improved from 20/60 (pinhole) to 20/50 (pinhole). In his left eye his visual acuity also improved, from 20/200 to 20/160 (with same correction). In his left eye, he continued to have a central dark spot due to macular scarring.

After eight months, Subject 4's vision in his right eye improved from 20/50 (pinhole) to 20/40 (pinhole) In his left eye his visual acuity improved from 20/160 to 20/100 (with same correction). The dark spot in the left eye started dissipating, and he could read hazily through the formerly dark spot. At this time his contrast sensitivity was also measured. His cataracts were measured at a 4+ (on a scale of 0-4, 4 being the highest). The central macular scar was barely visible to the ophthalmologist due to haziness of the optical path. After 10 months, Subject 1's visual acuity further improved from 20/15 to 20/10 (with the same correction).

After further 2 months; i.e., after a total of 12 months, Subject 4's vision continued to improve. The subject could now read books, newspapers, and the computer screen without any problems. The subject also showed improvement in cataracts (went from 4+ to 3-4+ on a 0-4 scale). The optical path clarity had improved enough that the macular scar was clearly visible to the ophthalmologist. In contrast sensitivity there was a 40% to 100% improvement. In Snellen acuity, he went from 20/40 to 20/30 (pinhole) in his right eye, and from 20/100 to 20/80 in his left eye.

### EXAMPLE 3

A second eye drop formulation of the invention, Formulation 2, was prepared as follows: High purity de-ionized (DI) water (500 ml) was filtered via a 0.2 micrometer filter. MSM (13.5 g), EDTA (6.5 g), and L-carnosine (5.0 g) were added to the filtered DI water, and mixed until visual transparency was achieved, indicating dissolution. The mixture was poured into 10 mL bottles each having a dropper cap. On a weight percent basis, the eye drop composition had the following components:

| | |
|---|---|
| Purified de-ionized water | 95.24 wt. % |
| MSM | 2.57 wt. % |
| Di-sodium EDTA | 1.24 wt. % |
| L-Carnosine | 0.95 wt. % |

### EXAMPLE 4

Subsequent to the experimentation described in Example 2, a detailed and controlled follow-on study was carried out using a slightly weaker eye drop formulation, Formulation 2 (prepared as described in Example 3). Placebo eye drops were also prepared and administered. The placebo drops were composed of a commercially obtained sterile saline solution in the form of a buffered isotonic aqueous solution (containing boric acid, sodium borate, and sodium chloride with 0.1 wt. % sorbic acid and 0.025 wt. % di-sodium EDTA as preservatives).

The study was double-masked, in that except for one positive control, neither the patient nor the ophthalmologist knew whether they were given the formulation eye drops or a saline solution. The patients were randomized to receive either the study formulation or saline solution.

The study involved five subjects, of which 3 subjects were given the eye drops of Formulation 2 and 1 subject was given placebo eye drops. In addition, 1 subject was given the higher-strength eye drops of Formulation 1. One drop (approximately 0.04 mL) was administered to each eye, two to four times daily for a period of 8 weeks. The drops were administered to both eyes of each subject. The study participants were multiethnic and 20% female, 80% male.

The baseline and follow-on testing by the ophthalmologist included: automated refraction; corneal topography; external photographs; wavefront photographs; visual acuity with spectacle correction at distance and at 14 inches; contrast sensitivity testing using the Vision Sciences Research Corporation (San Ramon, Calif.) Functional Acuity Contrast Test (FACT) chart; pupil examination and pupil size measurement; slit lamp examination; intraocular pressure measurement; and dilated fundus examination.

After 8 weeks, the subjects were examined again. The contrast sensitivity results for each subject are shown in Table 1, and all the results are summarized in Table 2.

TABLE 1

| | | Subject | | | | | | | | | |
|---|---|---|---|---|---|---|---|---|---|---|---|
| | | 1 | | 2 | | 3 | | 4⁵ | | 5⁶ | |
| | | Right (R) or Left (L) Eye | | | | | | | | | |
| Contrast Sensitivity (CS)¹ | | R | L | R | L | R | L | R | L | R | L |
| 1.5 cpd² | log₁₀ CS before | 1.85 | 1.56 | 1.70 | 1.70 | 2.00 | 1.85 | 1.56 | 1.70 | 1.85 | 1.70 |
| | log₁₀ CS after | 2.00 | 1.85 | 1.85 | 1.85 | 2.00 | 1.85 | 1.85 | 1.85 | 1.85 | 1.56 |
| | log₁₀ unit change³ | 0.15 | 0.29 | 0.15 | 0.15 | 0.00 | 0.00 | 0.29 | 0.15 | 0.00 | −.14 |
| | percent improved⁴ | 8 | 19 | 9 | 9 | 0 | 0 | 19 | 9 | 0 | −8 |
| 3 cpd | log₁₀ CS before | 1.90 | 1.76 | 1.90 | 1.90 | 1.90 | 1.90 | 1.76 | 1.90 | 1.76 | 1.90 |
| | log₁₀ CS after | 2.06 | 1.90 | 1.90 | 2.06 | 2.06 | 2.06 | 2.20 | 2.06 | 1.90 | 1.76 |
| | log₁₀ unit change | 0.16 | 0.14 | 0.00 | 0.16 | 0.16 | 0.16 | 0.44 | 0.16 | 0.14 | −.14 |
| | percent improved | 8 | 8 | 0 | 8 | 8 | 8 | 26 | 8 | 8 | −8 |
| 6 cpd | log₁₀ CS before | 1.81 | 1.81 | 1.95 | 2.11 | 1.95 | 1.95 | 1.65 | 1.81 | 1.81 | 1.81 |
| | log₁₀ CS after | 1.95 | 1.81 | 2.11 | 1.95 | 2.11 | 2.11 | 2.11 | 2.11 | 1.81 | 1.95 |
| | log₁₀ unit change | 0.14 | 0.00 | 0.16 | −.16 | 0.16 | 0.16 | 0.46 | 0.30 | 0.00 | 0.14 |
| | percent improved | 8 | 0 | 8 | −7 | 8 | 8 | 27 | 17 | 0 | 8 |
| 12 cpd | log₁₀ CS before | 1.34 | 1.18 | 1.78 | 1.78 | 1.78 | 1.78 | 1.18 | 1.48 | 1.48 | 1.63 |
| | log₁₀ CS after | 1.63 | 1.48 | 1.78 | 1.78 | 1.78 | 1.78 | 1.93 | 1.78 | 1.63 | 1.48 |
| | log₁₀ unit change | 0.29 | 0.30 | 0.00 | 0.00 | 0.00 | 0.00 | 0.75 | 0.30 | 0.15 | −.15 |
| | percent improved | 22 | 26 | 0 | 0 | 0 | 0 | 64 | 20 | 11 | −10 |
| 18 cpd | log₁₀ CS before | 0.90 | 1.08 | 1.23 | 1.23 | 1.23 | 1.30 | 0.60 | 1.23 | 0.90 | 1.23 |
| | log₁₀ CS after | 1.36 | 1.36 | 1.36 | 1.36 | 1.52 | 1.52 | 1.66 | 1.52 | 1.36 | 1.36 |
| | log₁₀ unit change | 0.46 | 0.28 | 0.13 | 0.13 | 0.29 | 0.22 | 1.06 | 0.29 | 0.46 | 0.13 |
| | PERCENT IMPROVED | 51 | 27 | 11 | 11 | 23 | 12 | 176 | 23 | 51 | 11 |

¹Contrast sensitivity (CS) is the reciprocal of the contrast at threshold, i.e., one divided by the lowest contrast at which forms or lines can be recognized. Log of the contrast sensitivity values is a generally accepted method for comparing contrast sensitivities.
²cpd = cycles per degree for the spatial frequency
³Log unit change = log₁₀(CS after treatment) − log₁₀(CS before treatment)
⁴Percent improved = [log₁₀(CS after treatment)/log₁₀(CS before treatment) − 1] × 100
⁵Positive control
⁶Placebo

TABLE 2

| | | Formu- | Formu- | |
|---|---|---|---|---|
| | | lation 1 | lation 2 | Saline |
| | | (positive | (study | Solution |
| | | control) | subjects) | (placebo) |
| | | n = 1 | n = 3 | n = 1 |
| PUPIL DILATION | | +20% | +8% | 0% |
| Snellen Acuity (distance vision) | | +17.5% | +7.5% | −15% |
| Snellen Acuity (near vision) | | 0% | +10% | 0% |
| Auto refraction | | +8% | +8% | 0% |
| Contrast Sensitivity¹ | | | | |
| 1.5 cpd² | percent improved³ | 14% | 7.5% | −4% |
| | log unit change⁴ | 0.22 | 0.12 | −0.08 |
| 3 cpd | percent improved | 17% | 6.8% | 0% |
| | log unit change | 0.33 | 0.12 | 0 |
| 6 cpd | percent improved | 22% | 4% | 4% |
| | log unit change | 0.38 | 0.08 | 0.08 |
| 12 cpd | percent improved | 42% | 7.9% | 0% |
| | log unit change | 0.53 | 0.10 | 0 |
| 18 cpd | percent improved | 99.5% | 22.2% | 31.0% |
| | log unit change | 0.68 | 0.24 | 0.26 |

| | | | |
|---|---|---|---|
| Wavefront (image tightness) | +23% | +38% | 0% |

¹Contrast sensitivity (CS) is the reciprocal of the contrast at threshold, i.e., one divided by the lowest contrast at which forms or lines can be recognized. Log of the contrast sensitivity values is a generally accepted method for comparing contrast sensitivities.
²cpd = cycles per degree for the spatial frequency
³Percent improved = [log₁₀(CS after treatment)/log₁₀(CS before treatment) − 1] × 100
⁴Log unit change = log₁₀(CS after treatment) − log₁₀(CS before treatment)

Subjects treated with Formulation 1 and Formulation 2 all showed very significant improvements, including improved smoothness and regularity of the cornea, improved accommodative/focusing ability, more uniform and stable tear film, and decreased yellowing of the cornea and lens. Subjects to whom the placebo was given did not exhibit any significant change. All subjects reported improved ability to see road signs at a distance, brighter and more vivid colors, and improved night vision.

### EXAMPLE 5

Formulation 1 was evaluated for efficacy in a 46-year-old male subject. Prior to treatment, the subject had no severe visual problems or eye abnormalities, but he did require bifocals to correct refractive errors in both eyes.

The subject was examined by an independent ophthalmologist prior to treatment and again following eight weeks of treatment. Tests performed included: Snellen visual acuity examinations for distance (20 feet) and near (14 inches) vision, autorefraction, pupil dilation (pupillometer maximum scotopic pupil size), slit lamp examination, automated corneal topography mapping, contrast sensitivity (functional acuity contrast test), automated wavefront aberration mapping, and photographs of the anterior segment.

Treatment consisted of the topical instillation of one drop (approximately 0.04 mL) of Formulation 1 in each eye two to four times per day for eight weeks. Results of this treatment were as follows:

No irritation, redness, pain, or other adverse effects were observed by the ophthalmologist or reported by the subject, other than transient minor eye irritation at the time of eye drop administration.

Snellen visual acuity: Using the same refractive correction, distance visual acuity improved from 20/25+1 to 20/20 in the right eye, and from 20/20-2 to 20/20 in the left eye. Near vision was unchanged at 20/50 in both eyes.

Autorefraction: The right eye was unchanged: spherical −3.75; astigmatism +2.5 at axis of 24 degrees. The left eye showed slight improvement: spherical decreased from −4.00 to −3.75; astigmatism decreased from +3.50 at 175 degrees to +3.25 at 179 degrees.

Pupil dilation: Both eyes improved from 5.0 to 6.0 mm.

Slit lamp examination: The retinas appeared unchanged, and no cataracts were observed during either examination.

Corneal topography: Improved smoothness and regularity of the cornea were observed in both eyes. The ophthalmologist remarked that the improvement may have been due to a more uniform and stable tear film.

Contrast sensitivity: Measurements are shown in Table 3.

TABLE 3

| CPD* | | | | |
|---|---|---|---|---|
| 1.5 | 3 | 6 | 12 | 18 |

Eye

| | R | L | R | L | R | L | R | L | R | L |
|---|---|---|---|---|---|---|---|---|---|---|
| Before | 6 | 7 | 6 | 7 | 5 | 6 | 3 | 5 | 1 | 5 |
| After | 8 | 8 | 9 | 8 | 8 | 8 | 8 | 7 | 8 | 7 |

*CPD = cycles per degree (spatial frequency of pattern)

These data indicate a consistent, significant improvement in contrast sensitivity.

Automated wavefront mapping: For the right eye, spherical aberration was essentially uncharged (+0.15660 to +0.15995). Retinal image formation improved from 60×70 to 45×70 minutes of arc, which represents a 25% tighter image formation. For the left eye: Spherical aberration decreased from +0.14512 to +0.09509, representing a 34.4% improvement. Retinal image formation improved with an estimated 20% tighter image.

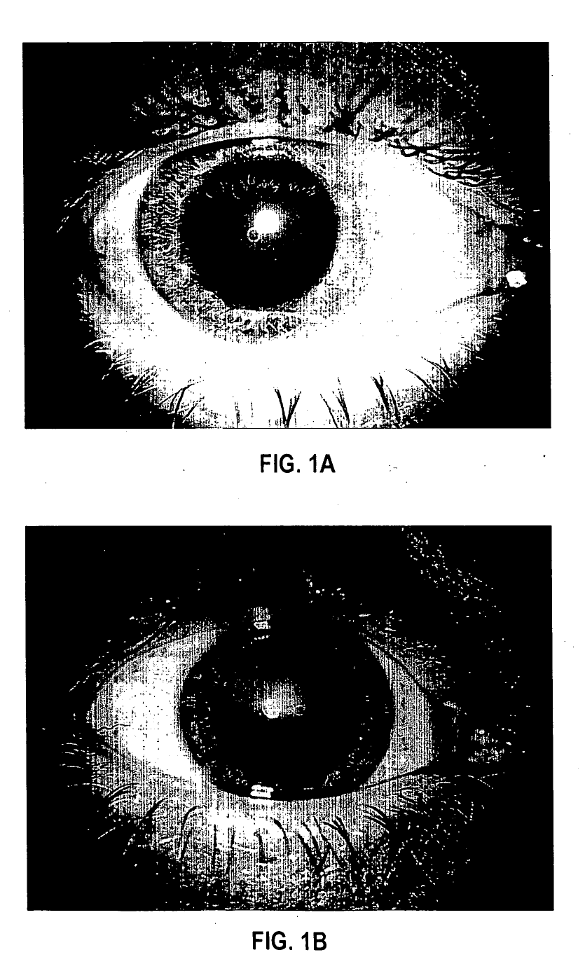

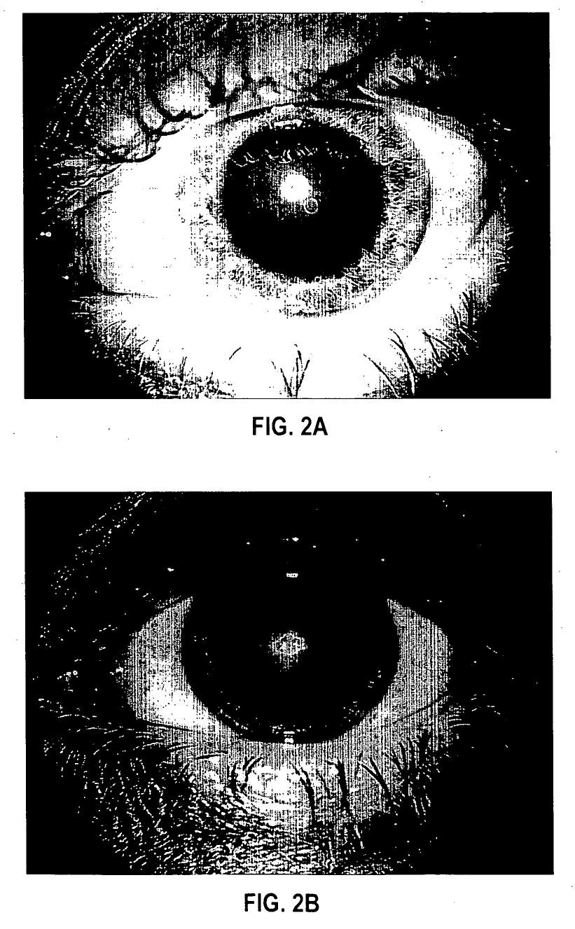

Photographs of anterior segment, FIG. 1A (OD, before treatment), FIG. 1B (OD, after treatment), FIG. 2A (OS, before treatment), and FIG. 2B (OS, after treatment): Iris color changed to a darker blue; the degree of change was reported as “striking.” The change was likely due to a decrease in the yellowing of the cornea.

In addition, the subject reported that, following treatment, he switched to lower power prescription glasses and no longer required bifocals. He made the following remarks: “I have been using the eye drops for about eight weeks, and my eyesight has significantly improved. I can see colors more vividly. I have replaced my bifocals with my older, lower power non-bifocals. I can see much better in the distance and do not need reading glasses. My eyes have become a darker blue like my original eye color, and my night vision has improved.”

### EXAMPLE 6

Formulation 1 was evaluated for efficacy in a 60-year-old male subject. Prior to treatment, the subject had no serious visual problems or eye abnormalities other than refractive errors in both eyes.

The subject was examined by an independent ophthalmologist prior to treatment and again following seven weeks of treatment. Tests performed included: Snellen visual acuity examinations for distance (20 feet) and near (14 inches) vision, autorefraction, pupil dilation (pupillometer maximum scotopic pupil size), slit lamp examination, automated corneal topography mapping, contrast sensitivity (functional acuity contrast test), automated wavefront aberration mapping, and photographs of the anterior segment.

Treatment consisted of the topical instillation of one drop (approximately 0.04 mL) of Formulation 1 in each eye two to four times per day for seven weeks. Results of this treatment were as follows:

No irritation, redness, pain, or other adverse effects were observed by the ophthalmologist or reported by the subject, other than transient minor eye irritation at the time of eye drop administration.

Snellen visual acuity: Using the same refractive correction (intentionally undercorrected in the left eye), distance visual acuity remained unchanged at 20/15 in the right eye, and improved from 20/40-2 to 20/40 in the left eye. Near vision declined from 20/70 to 20/100 in the right eye (likely due to overcorrection for distance), and improved from 20/40-2 to 20/25 in the left eye.

Autorefraction: The right eye had an unchanged spherical measurement

(−6.00) and a slight improvement in astigmatism (+0.75 at 115 degrees to +0.50 at 113 degrees). The left eye showed slight improvement: spherical went from −8.25 to −8.00; astigmatism was unchanged, from +1.00 at 84 degrees to +1.00 at 82 degrees.

Pupil dilation: The right eye improved from 4.0 to 4.5 mm, and the left eye was unchanged at 4.0 mm.

Slit lamp examination: The retinas appeared unchanged, and minimal cataracts were observed during both examinations.

Corneal topography: Improved smoothness and regularity of the cornea were observed in both eyes. The ophthalmologist remarked that the improvement may have been due to a more uniform and stable tear film.

Contrast sensitivity: Measurements are shown in Table 4.

TABLE 4

| | CPD* | | | | | | | | | |
|---|---|---|---|---|---|---|---|---|---|---|
| | 1.5 | | 3 | | 6 | | 12 | | 18 | |
| | Eye | | | | | | | | | |
| | R | L | R | L | R | L | R | L | R | L |
| Before | 8 | 6 | 7 | 6 | 6 | 6 | 4 | 3 | 3 | 4 |
| After | 9 | 8 | 8 | 7 | 7 | 6 | 6 | 5 | 6 | 6 |

*CPD = cycles per degree (spatial frequency of pattern)

These data indicate a consistent, significant improvement in contrast sensitivity.

Automated wavefront mapping: For the right eye: Spherical aberration decreased from +0.01367 to +0.00425, a 69% improvement. Retinal image formation improved from 80×80 to 70×65 minutes of arc, which represents a 28.9% tighter image formation. For the left eye: Spherical aberration decreased from +0.04687 to −0.00494, representing a >100% improvement. Retinal image formation improved from 150×150 to 100×100 minutes of arc, which represents a 33% tighter image formation. The ophthalmologist remarked at the second examination: “Overall spherical aberration is closer to that of a young healthy eye rather than a 60-year-old eye.”

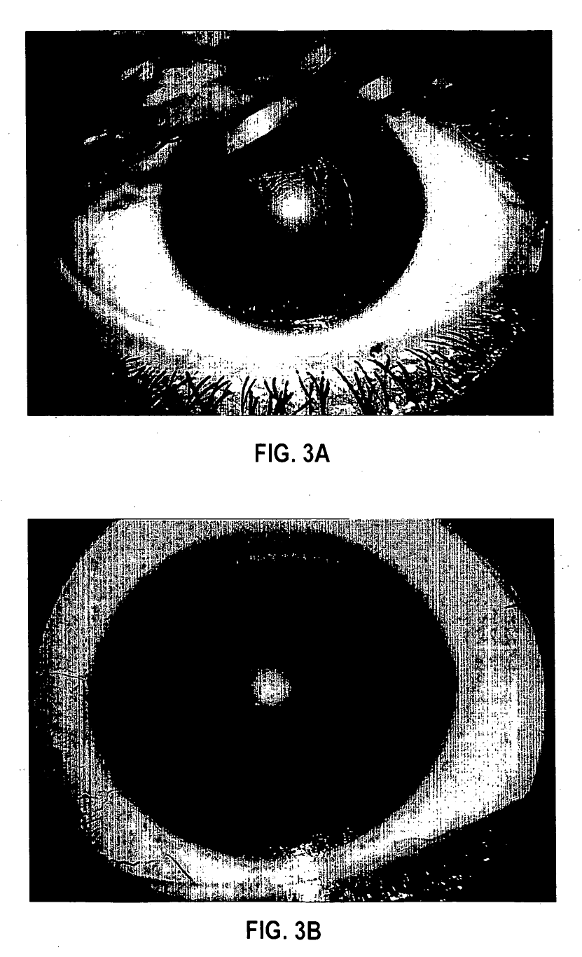

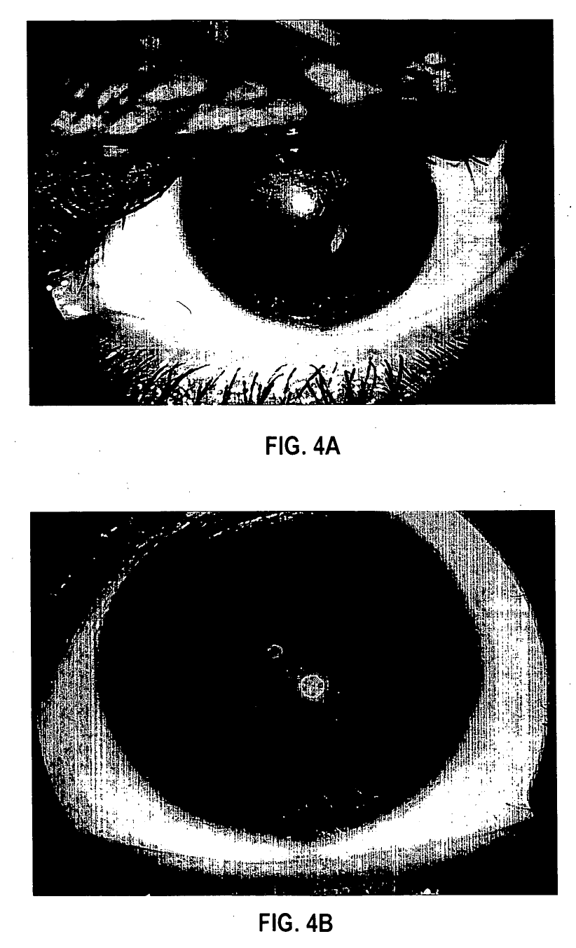

Photographs of anterior segment, FIG. 3A (OD, before treatment), FIG. 3B (OD, after treatment), FIG. 4A (OS, before treatment), and FIG. 4B (OS, after treatment): Observed were an apparent decrease in lens opacity, reduced yellowing of the crystalline lens, and improved corneal clarity.

In addition, the subject stated: “I have used these eye drops for about seven weeks. I can see a golf ball at 300 yards, whereas it was barely visible at 220 yards before. My vision vastly improved, especially in seeing road signs in the distance. I see colors much more brightly and vividly.”

### EXAMPLE 7

The ocular pharmacokinetic behavior of EDTA, when administered as a component of Formulation 1, was evaluated in rabbits over a period of five days. Two healthy male rabbits, each approximately 2.5 to 3 kg in body weight, were used for the study.

On day 1 of the study, one drop of Formulation 1 was topically instilled in each eye of both rabbits (four eyes total). No additional eye drops were administered during the course of the study. Samples of aqueous humor were extracted at 15 min, 30 min, 1 hr, 4 hrs, 3 days, and 5 days following administration (as indicated in the following table). Vitreous humor was extracted at 5 days following administration from all four eyes. The concentration of EDTA was measured in all the samples of aqueous humor and vitreous humor by HPLC analysis.

The results of the study are summarized in Table 5.

TABLE 5

| | Concentration of EDTA (micrograms per milliliter) | | | |
|---|---|---|---|---|
| | Rabbit 101 | | Rabbit 102 | |
| | Right Eye | Left Eye | Right Eye | Left Eye |
| Aqueous humor: | | | | |
| 15 min | 1.3 | | | |
| 30 min | | | 10.7 | |
| 1 hr | | 5.3 | | |
| 4 hrs | | | | 0.9 |
| 3 days | 0.5 | 0.4 | 0.5 | 0.7 |
| 5 days | 0.6 | 0.5 | 0.4 | 0.6 |
| Vitreous humor: | | | | |
| 5 days | 0.6 | 0.5 | 0.7 | 0.6 |

Examples 1-7 indicate that topical drops composed of the multifunction agents MSM and EDTA, with the addition of the L-carnosine AGE breakers, significantly improved the quality of both day and night vision (visual acuity), greatly improved contrast sensitivity, improved pupil dilation, produced a more uniform and stable tear film, reduced arcus senilis, and greatly reduced glare and the discomfort associated with photophobia. No adverse pathological changes or reduction in acuity were observed.

### EXAMPLE 8

In the following in vivo experiment, the ocular pharmacokinetic behavior of EDTA, when administered with MSM as permeation enhancing penetrating agent, was evaluated in rabbits over a period of five days. Two healthy male rabbits, each approximately 2.5 to 3 Kg in body weight, were used for the study.

An eye drop formulation of the invention, was prepared as follows: High purity de-ionized (DI) water (500 ml) was filtered via a 0.2 micron filter. MSM (27 g), EDTA (13 g), and L-carnosine (5 g) were added to the filtered DI water, and mixed until visual transparency was achieved, indicating dissolution. The mixture was poured into 10 ml bottles each having a dropper cap. On a weight percent basis, the eye drops had the following composition:

| | |
|---|---|
| Purified de-ionized water | 91.74 wt. % |
| MSM | 4.95 wt. % |
| Di-sodium EDTA | 2.39 wt. % |
| L-Carnosine | 0.92 wt. %. |

On day 1 of the study, one drop of Formulation 1 was topically instilled in each eye of both rabbits (four eyes total). No additional eye drops were administered during the course of the study. Samples of aqueous humor were extracted at 15 min, 30 min, 1 hr, 4 hrs, 3 days, and 5 days following administration (as indicated in the following table). Vitreous humor was extracted at 5 days following administration from all four eyes. The concentration of EDTA was measured in all the samples of aqueous humor and vitreous humor by HPLC analysis.

The results of the study are summarized in the following table:

| | Concentration of EDTA (micrograms per milliliter) | | | |
|---|---|---|---|---|
| | Rabbit 101 | | Rabbit 102 | |
| | Right Eye | Left Eye | Right Eye | Left Eye |
| Aqueous humor: | | | | |
| 15 min | 1.3 | | | |
| 30 min | | | 10.7 | |
| 1 hr | | 5.3 | | |
| 4 hrs | | | | 0.9 |
| 3 days | 0.5 | 0.4 | 0.5 | 0.7 |
| 5 days | 0.6 | 0.5 | 0.4 | 0.6 |
| Vitreous humor: | | | | |
| 5 days | 0.6 | 0.5 | 0.7 | 0.6 |

These results show that Formulation 1 delivers EDTA to the anterior chamber of the eye (aqueous humor) very rapidly: a concentration of 10.7 μg/mL is reached at only 30 minutes following administration. Because the aqueous humor is completely flushed from the anterior chamber approximately every 90 minutes, compounds from conventional eye drop formulations are typically not detected in the aqueous humor at four hours following administration. We, however, observed significant concentrations of EDTA in the aqueous humor even at five days following administration. Our data also show that EDTA reached the vitreous humor, where it was present in almost the same concentration as in the aqueous humor. It is thus likely that the vitreous humor (and probably adjacent tissues) was acting as a reservoir for the absorbed EDTA, with some of this EDTA diffusing back into the aqueous humor over time.

The demonstrated penetration of EDTA from Formulation 1 into the posterior segment of the eye, including the vitreous humor, indicates the potential of the inventive formulation to deliver therapeutic agents to the posterior of the eye when administered as eye drops. Such drug delivery to the posterior of the eye allows for the treatment of many eye conditions, diseases, and disorders, including age related macular degeneration, macular edema, glaucoma, cell transplant rejection, infections, and uveitis.

### EXAMPLE 10

Formulation 1 was evaluated for efficacy in treating a male subject in his eighties who was suffering from cataracts and Salzmann's nodules, whose best correction had been 20/400 in his left eye and 20/200 in his right eye, and had acute photophobia and glare, as well as severe macular scarring in the left eye. The formulation was administered to the subject, one drop (approximately 0.04 ml) to each eye, two to four times per day for a period of over 12 months. There were no side effects, other than minor temporary irritation at the time of administering the formulation in the eye, were reported or observed by the subject or the ophthalmologist.

After 4 weeks into the study, the changes reported by the subject included increased brightness, improved clarity of vision, and reduced glare. After 8 weeks the glare and photophobia were reduced, and the subject started to read books, newspapers, and information on the computer screen again. The visual acuity in his right eye improved significantly, from 20/200 (with correction) to 20/60 (pinhole) (with the same correction). In his left eye, his visual acuity improved as well, from 20/400 to 20/200 (with the same correction). In his left eye, he continued to have a central dark spot due to macular scarring.

The subject reported a further reduction in glare and photophobia, and further improvements in the ease of reading books, newspapers, and information on the computer screen. Subject also reported that nighttime glare had been eliminated. The subject was now comfortable in daylight without need for dark glasses, and without suffering severe problems with glare. The visual acuity in his right eye improved from 20/60 (pinhole) to 20/50 (pinhole) In his left eye his visual acuity also improved, from 20/200 to 20/160 (with same correction). In his left eye, he continued to have a central dark spot due to macular scarring.

After eight months, the subject's vision in his right eye improved from 20/50 (pinhole) to 20/40 (pinhole) In his left eye his visual acuity improved from 20/160 to 20/100 (with same correction). The dark spot in the left eye started dissipating, and he could read hazily through the formerly dark spot. At this time his contrast sensitivity was also measured. His cataracts were measured at a 4+ (on a scale of 0-4, 4 being the highest). The central macular scar was barely visible to the ophthalmologist due to haziness of the optical path.

After a total of 12 months, the subject's vision continued to improve. The subject could now read books, newspapers, and the computer screen without any problems. The subject also showed improvement in cataracts (went from 4+ to 3-4+ on a 0-4 scale). The optical path clarity had improved enough that the macular scar was clearly visible to the ophthalmologist. In contrast sensitivity there was a 40% to 100% improvement. In Snellen acuity, from 20/40 to 20/30 (pinhole) in his right eye, and from 20/100 to 20/80 in his left eye. The subject also reported that for the first time in 40 years he could start to see wavy letters through his left eye.

These results demonstrate that the eye-drops are reaching the retina in the back of the eye, and the MSM was aiding the penetration of EDTA and L-Carnosine. These results are consistent with the rabbit study of Example 4.

### EXAMPLE 11

Formulation 1 was evaluated for efficacy in treating a female subject in her sixties who was having problems with “floaters” in both of her eyes. The formulation was administered to the subject, one drop (approximately 0.04 ml) to each eye, two to four times per day for a period of over 12 months. There were no side effects, other than minor temporary irritation at the time of administering the formulation in the eye, were reported or observed by the subject or the ophthalmologist.

After 8 weeks of using the eye drops, the subject reported a significant reduction in the floaters, again confirming that medication was reaching the vitreous, and having a beneficial effect.

### EXAMPLE 12

Formulation 1 was evaluated for efficacy in treating a male subject in his fifties who was had a visual acuity of 20/15 with correction and a very prominent arcus senilis. The formulation was administered to the subject, one drop (approximately 0.04 ml) to each eye, two to four times per day for a period of over 12 months. There were no side effects, other than minor temporary irritation at the time of administering the formulation in the eye, were reported or observed by the subject or the ophthalmologist.

After 16 weeks, the subject reported improvement in visual acuity from 20/25 to 20/15, as well as very significant reduction in his arcus senilis.

### EXAMPLE 13

An eye drop formulation of the invention, Formulation 3, was prepared as follows: Approximately 500 ml of high purity de-ionized (DI) water was filtered via a 0.2 micrometer filter and 27 g of Methylsulfonylmethane (MSM), and 13 g of Ethylenediaminetetraacetic acid disodium salt, dihydrate (EDTA) were added. The formulation was mixed until visual transparency was achieved, the pH was adjusted to 7.2 with NaOH, and the volume was adjusted to 500 ml. The mixture was poured into 10 mL bottles each having a dropper cap. On a weight percent basis, the eye drops had the following composition:

| | |
|---|---|
| Purified de-ionized water | 92.0 wt. % |
| MSM | 5.40 wt. % |
| EDTA disodium salt, dihydrate | 2.60 wt. % |

Formulation 3 was evaluated for efficacy for a maximum period of 120 days. Patients were given either Formulation 3 or the placebo (commercially available unpreserved saline) and instructed to use one drop (approximately 0.04 ml) to each eye, four times per day. The patients were randomized to receive either the study formulation or the placebo. Twelve eyes received Formulation 3 while thirteen eyes received the placebo. The study was double-masked, in that neither the patient nor the ophthalmologist knew whether they were given Formulation 3 eye drops or the placebo.

Contrast sensitivity was measured under mesopic conditions simulating dusk (3 candles/m²) using the FACT™ (Functional Acuity Contrast Test) and a CST 1800 Digital® contrast sensitivity tester. Measurements were performed monocularly, in duplicate, for each eye and duplicate measurements were averaged.

The FACT™ uses a sine-wave grating chart to test for contrast sensitivity. The chart consists of five rows (spatial frequencies), each row having nine levels of contrast sensitivity. Sine wave gratings are special test patterns that appear as varying sizes and contrasts of gray bars set up in circular patterns. The gratings in spatial frequency A appear as the largest gray bars (longest wavelength) while the gratings in spatial frequency E appear as the smallest gray bars (shortest wavelength). While viewing the chart through the CST 1800 Digital® contrast sensitivity tester, subjects report the orientation of each grating: right, up or left. For each spatial frequency, there are nine levels of contrast sensitivity, also called patches. Level 1 has the greatest contrast, while level 9 has the least. The subject reports the orientation of the last grating seen (1 through 9) for each row (A, B, C, D and E).

When the FACT is scored, the nine levels of contrast sensitivity are graphed using a logarithmic scale. An improvement of one level or patch represents approximately a 1.5-fold increase in contrast sensitivity. To quantify the contrast sensitivity improvement, data from Day 14 (T₀) were compared to the last contrast sensitivity data obtained for each subject that completed at least 60 days of treatment.

Of the twelve eyes that received Formulation 3, eight eyes (67%) showed a contrast sensitivity improvement of at least two patches in two spatial frequencies, a statistically significant result (p=0.0237). Of the thirteen eyes that received the placebo, only three (23%) showed an improvement of at least two patches in two spatial frequencies.

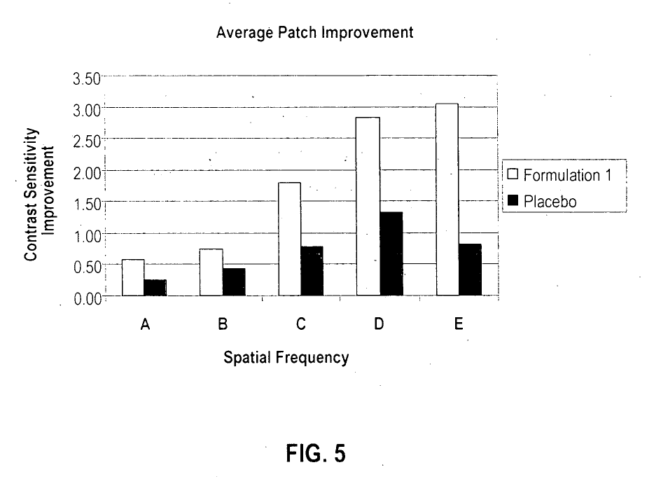

As another measure of contrast sensitivity improvement, the average patch improvement of the eyes that received Formulation 3 was compared to the group of eyes that received the placebo for each spatial frequency (FIG. 5). The eyes that received Formulation 3 showed a significant contrast sensitivity improvement in all spatial frequencies, with an improvement of greater than 2.5 patches in spatial frequency D and an improvement of over 3 patches for spatial frequency E.

None of the subject reported serious ocular or systemic adverse events.

### EXAMPLE 15

Objective. Determine the extent of penetration of ¹⁴C-EDTA into the aqueous of the eye, with and without MSM present, in eye drops applied to rat eyes.

Reagents. Ethylenediamine tetraacetic acid-1,2-¹⁴C tetrasodium was purchased from Sigma. ¹⁴C-EDTA (Specific Activity: 10.6 mCi/mmol, radiochemical purity: 99% or higher). All other chemicals used in this study were of analytical grade and purchased commercially. ScintiVerse II Cocktail (Liquid Scintillation Solvent) was general-purpose LSC Cocktail for aqueous, non-aqueous, and emulsion counting systems from Fisher Scientific.

Animals. Male Sprague-Dawley rats weighing 200-250 g were obtained from Central Animal Care Services at the University of Texas Medical Branch. The NIH guidelines and ARVO statement for the Use of Animals in Ophthalmic and Vision Research were strictly followed for the welfare of the animals. Rats were sacrificed using 100% carbon dioxide at a low flow rate (25-30% of the volume of the cage per minute) for about 2 minutes.

Experimental Procedure. 100 μl of each of the following three eye drop solutions were prepared.

Solution A

80 μl of 5.4% MSM

10 μl of 600 mM EDTA (Tetrasodium salt EDTA)

10 μl of C¹⁴ EDTA (Directly from the bottle)

Solution B:

80 μl of 5.4% MSM

10 μl of 120 mM EDTA (Tetrasodium salt EDTA)

10 μl of C¹⁴ EDTA (Directly from the bottle)

Solution C:

80 μl of PBS

10 μl of 600 mM EDTA (Tetrasodium salt EDTA)

10 μl of C¹⁴ EDTA (Directly from the bottle)

8 μl of each eye drop solution was applied to the cornea of each of the eyes. One rat was treated with each solution. At 0.5, 2, and 16 hours, aqueous humor was aspirated from each eye using a 30-gauge fine needle with an Insulin-syringe and dispensed in 50 μl of PBS. To solubilize the protein, samples were placed in a 50° C. water bath for 3 hours followed by centrifugation at 10,000 rpm for 10 minutes.

Determination of the radioactivity of the samples. Samples were added to the counting vials containing 25 ml of ScintiVerse II counting fluid, mixed vigorously and allowed to stand for 1 hour in the dark. The samples were then counted using a Liquid Scintillation counter (LS 1801 Liquid Scintillation Systems, Beckman Instruments, Inc.). Counts per minute were averaged for the two eyes that received each solution for each time point.

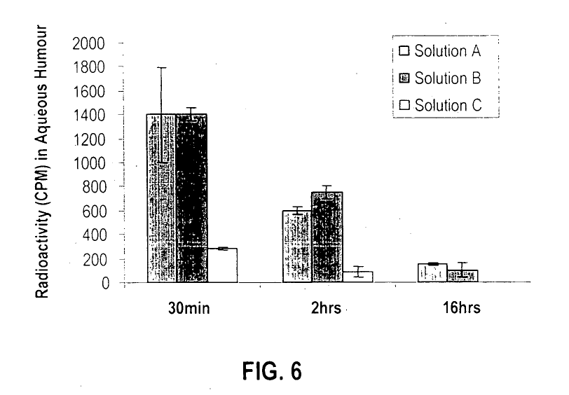

To evaluate the ability of each solution to be transported from the cornea to the aqueous humor, the amount of ¹⁴C-EDTA in the aqueous humor was compared between Solutions A, B, and C (FIG. 6). In the absence of MSM, very little EDTA was present in the aqueous humor, regardless of the EDTA concentration. At the 30-minute time point, there was an increase of approximately 5-fold in the amount of ¹⁴C-EDTA in the aqueous humor in the presence of MSM.

### EXAMPLE 16

### EDTA Pharmacokinetic Study

Objective. Determine the amount of C-14 labeled EDTA that penetrates into the various structures of the rat eye (cornea, aqueous humor, lens, vitreous, and retina), using eye drops that contain MSM. A comparison of two different eye drop formulations that differed in their EDTA concentration.

Reagents. Ethylenediaminetetra acetic acid-1,2-¹⁴C tetrasodium was purchased from Sigma. ¹⁴C-EDTA (Specific Activity: 10.6 mCi/mmol, radiochemical purity: 99% or higher). All other chemicals used in this study were of analytical grade and purchased commercially. ScintiVerse II Cocktail (Liquid Scintillation Solvent) was general-purpose LSC Cocktail for aqueous, nonaqueous and emulsion counting systems from Fisher Scientific.

Animals. Male Sprague-Dawley rats weighing 200-250 g were obtained from Central Animal Care Services at the University of Texas Medical Branch. The NIH guidelines and ARVO statement for the Use of Animals in Ophthalmic and Vision Research were strictly followed for the welfare of the animals. Rats were sacrificed using 100% carbon dioxide at a low flow rate (25-30% of the volume of the cage per minute) for about 2 minutes.

Eye Drop 1.

60.5 mM EDTA

10 μl, 5 μCi of ¹⁴C-EDTA;

10 μl of 600 mM EDTA;

80 μl of 5.4% MSM.

Eye Drop 2.

12.5 mM EDTA

10 μl, 5 μCi of ¹⁴C-EDTA;

10 μl of 120 mM EDTA;

80 μl of 5.4% MSM.

8 μl of Eye Drop 1 was applied to the rats' eyes. After 0.5, 1, 2, 4, and 16 hours, the rats were sacrificed and the eyeballs removed. The eyeballs were quickly washed 6 times in 5 ml of saline each time. Aqueous humor was aspirated from both eyes and dispensed in 50 μl of PBS. Cornea, lens, vitreous and retina from each eye were separated and placed in Eppendorf tubes containing H₂O and ION NaOH in the following ratio:

Cornea: 200 μl H₂O+40 μl of ION NaOH;

Lens: 500 μl H₂O+100 μl of ION NaOH;

Vitreous: 200 μl H₂O+40 μl of ION NaOH;

Retina: 200 μl H₂O+40 μl of ION NaOH.

To solubilize the protein, samples were placed in a 50° C. water bath for 3 hours followed by centrifugation at 10,000 rpm for 10 minutes. Samples were added to the counting vials containing 25 ml of ScintiVerse II counting fluid, mixed vigorously and allowed to stand for 1 hour in the dark. They were then counted using a Beckman Scintillation counter (LS 1801 Liquid Scintillation Systems, Beckman Instruments, Inc.).

8 μl of Eye Drop 2 was applied to the rat eye. After 0.5, 2 and 4 hours, the rats were sacrificed and the experiment conducted in the same way as for Eye Drop 1.

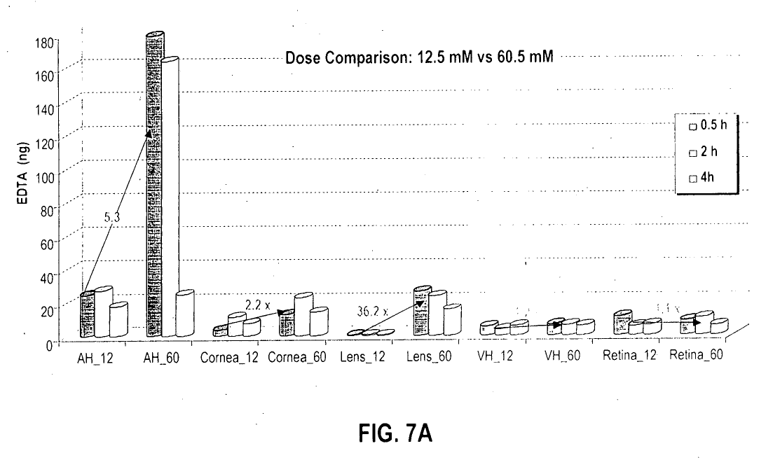

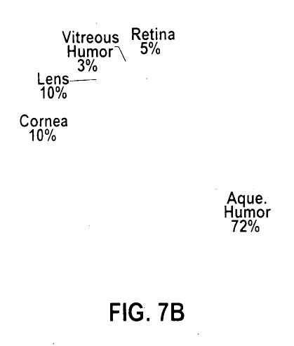

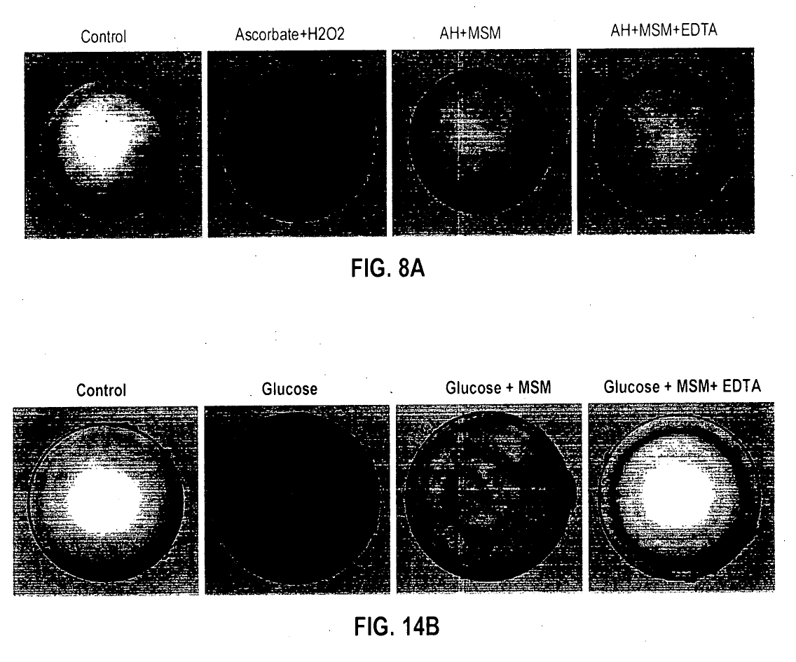

To look at the distribution of each formulation in the eye structures, the number of nanograms of EDTA was calculated for each time point (FIG. 7A). Dose dependency was observed, particularly in the aqueous humor, the cornea, and the lens. The percentage of EDTA found in each eye structure was calculated for the two-hour time point for Eye Drop 1 (FIG. 7B). The majority of the EDTA was found in the aqueous humor; however, the Eye Drop 1 formulation was present in all tissues examined.

### EXAMPLE 17

### Evaluation of Oxidation-Induced Toxicity in Rat Lens Organ Culture (RLCE)

Materials. EDTA, Ascorbic acid, and H₂O₂ were purchased from Sigma. All cell culture medium components were from Invitrogen.

Animals. Male Sprague-Dawley rats weighing 200-250 g were obtained from Central Animal Care Services at the University of Texas Medical Branch. The NIH guidelines and ARVO statement for the Use of Animals in Ophthalmic and Vision Research were strictly followed for the welfare of the animals. Rats were sacrificed with using 100% carbon dioxide at a low flow rate (25-30% of the volume of the cage per minute) for about 2 minutes.

Lens Culture. The rat lenses were dissected and washed with 1% penicillin/streptomycin in sterile PBS. The lenses were cultured in medium 199 containing 0.1% gentamicin at 37° C. in a 5% CO₂ humidified atmosphere. The lenses were divided into groups of two lenses each and were exposed to either glucose or ascorbate with H₂O₂ MSM and/or EDTA. The medium was changed every day for 7 days. The lenses were visualized under a Nikon Eclipse 200 and photographs were taken using a Multidimensional Imaging System.

Preparation of Reagents.

| | |
|---|---|
| Medium M199 + 0.1% | 250 ml of M199 + 250 μl of gentamicin |
| gentamicin | |
| 400 mM MSM (FW 94.2) | 376 mg MSM + PBS to final volume |
| | to 10 ml |
| 50 mM EDTA | 190 mg EDTA + PBS 8 ml, adjust |
| (Tetrasodium Salt FW 380) | pH to 7.2 with HCl, adjust final |
| | volume to 10 ml. |
| 2.5 M glucose (FW 180) | 900 mg glucose + 2 ml ddH₂O |
| 100 mM ascorbate (FW174) | 176 mg ascorbate + 10 ml ddH₂O |
| 10 mM H₂O₂ | 11 μl of 30% H₂O₂ + ddH₂O |
| | to final volume 10 ml. |

Experimental Procedure. 1. Sacrificed seven rats, removed the eyeballs as soon as possible, and put them into a tube containing PBS with 0.1% gentamicin. 2. Dissected the lenses immediately and washed with PBS. 3. Transferred all lenses to two 12-well plates (2 ml of medium per well/per lens). Each treatment was performed in 2 wells. Final concentrations for the six treatments were as follows:

50 mM glucose

50 mM glucose+4 mM MSM

50 mM glucose+4 mM MSM+0.5 mM EDTA

1 mM ascorbate+100 μM hydrogen peroxide

1 mM ascorbate+100 μM hydrogen peroxide+4 mM MSM

1 mM ascorbate+100 μM hydrogen peroxide+4 mM MSM+0.5 mM EDTA

4. The medium and the reagents were changed every day. 5. After 7 days of lens culture, took photographs and determined level of light transparency through the lenses.

Results. Photographs of the lens culture showed that significant rat lens opacity was induced with both glucose and ascorbate plus hydrogen peroxide (FIGS. 8A and 8B). MSM mitigated lens opacification by both oxidants; however, MSM plus EDTA provided the most effective protection.

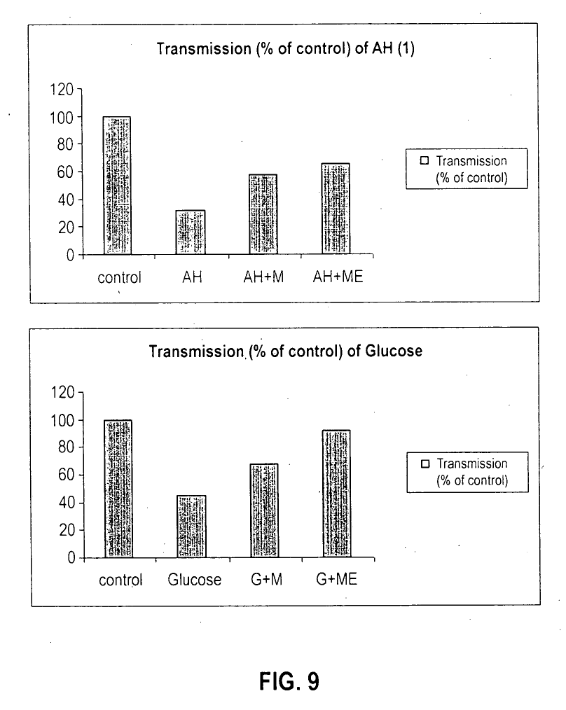

The level of light transmission through the lens was used to quantify lens opacity for each treatment. Consistent with the photographic results, MSM improved the level of light transmission for both oxidative treatments, while MSM+EDTA gave an even greater improvement (FIG. 9). Light transmission through the lens treated with ascorbate/hydrogen peroxide (AH) was 32% of light transmission through the control (upper graph). Light transmission through the lenses treated with ascorbate/hydrogen peroxide and MSM (AH+M) and ascorbate/hydrogen peroxide and MSM/EDTA (AH+ME) were 57% and 66% respectively. A similar pattern was observed when 50 mM Glucose was used as the oxidant (lower graph). Light transmission through the lens treated with glucose was only 45% of light transmission through the untreated control. Light transmission through the lenses treated with glucose plus MSM (G+M) and glucose and MSM/EDTA (G+ME) were 68% and 92% respectively.

### EXAMPLE 18

### Evaluation of Cell Viability Following Oxidation-Induced Toxicity in Human Lens Epithelial Cells (HLEC) and Protection with MSM and/or EDTA

Materials. EDTA (Tetrasodium Salt), Ferrous ammonium sulfate, Ferric chloride, Adenosine 5′-diphosphate (ADP), Ascorbic acid, and H₂O₂ were purchased from Sigma. All cell culture medium components were from Invitrogen.

Cell Culture and Treatment. Human lens epithelial cells (HLECs) with extended life span were cultured in DMEM medium containing 0.1% Gentamicin and supplemented with 20% fetal bovine serum at 37° C. in a 5% CO₂ humidified atmosphere. 10×10⁵ HLECs /ml (Passage 5) were seeded in 12-well plate overnight prior to the addition of oxidation reagents and MSM and/or EDTA.

Cell Viability. Cell survival was determined by Trypan Blue staining and counting with a hemocytometer. Dead cells stain blue, while live cells exclude Trypan Blue. Cell viability is represented as percentage of the number of live cells/number of total cells.

Preparation of Reagents.

| | |
|---|---|
| HLEC medium | DMEM + 20% FBS + 0.1% gentamicin |
| 400 mM MSM | 376 mg/10 ml PBS for stock |
| 50 mM EDTA | 190 mg/10 ml PBS for stock, pH 7.2 |
| (Tetrasodium Salt) | |
| Hydrogen peroxide | 30 mM stock |
| 5 M Glucose | 1800 mg/10 ml of ddH₂O |
| 100 mM Ascorbate | 176 mg/10 ml of ddH₂O |
| Fenton | Ferrous ammonium sulfate (FAS) 1 mM, |
| | ADP 10 mM, H₂O₂ 10 mM |
| Fenton' | FAS 1 mM, ADP 10 mM, H₂O₂ 10 mM |
| Ferric Chloride | FeCl₃ 5 mM, EDTA 5 mM, H₂O₂ 20 mM |

Experimental Procedure. 1. Seeded 0.5×10⁵/ml of HLEC (Passage 5) into three 12-well plates, incubated at 37° C. for overnight. 2. Changed medium to 2% FBS DMEM medium. 3. Added the oxidation reagents and MSM and/or EDTA to the proper wells. Final concentrations were as follows:

4 mM MSM

0.5 mM EDTA

100 μM H₂O₂

50 mM glucose

1 mM ascorbate

Fenton: Ferrous ammonium sulfate (FAS) 10 μM, ADP 100 μM, H₂O₂ 100 μM

Fenton′: FAS 10 μM, ADP 100 μM, H₂O₂ 100 μM

Ferric Chloride: FeCl₃ 50 μM, EDTA 50 μM, H₂O₂ 200 μM

After adding oxidation reagents and MSM and/or EDTA, cells were incubated at 37° C. with 5% CO₂ and 95% air for 16 hrs, and harvested with 0.25%Trypsin-EDTA and cell viability determined with Trypan-Blue.

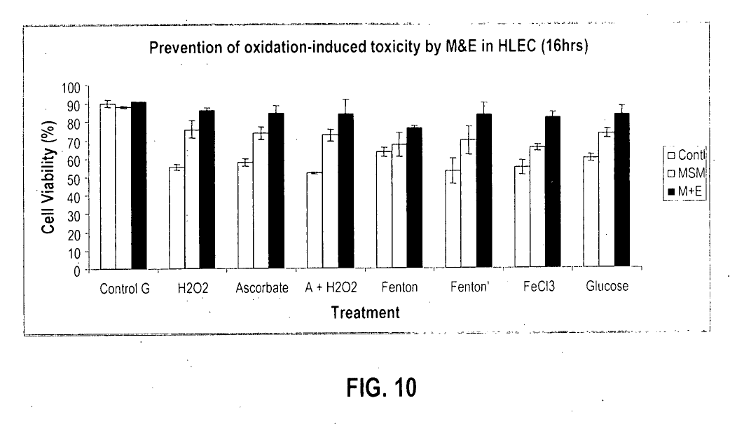

Results. FIG. 10 shows the percent of cell viability under each condition. The oxidants decreased cell viability between 30% (Fenton) and almost 45% (ascorbate+H₂O₂). The addition of 4 mM MSM increased the percent cell viability for all oxidants, while the addition of 4 mM MSM with 0.5 mM EDTA gave a greater increase in the percentage of viable cells. A Chi Square test was performed to determine whether the protective effects of MSM/EDTA were statistically significant. For those wells containing an oxidant plus the MSM/EDTA mixture, statistically significant results (P value of less than 0.05) were obtained for all oxidants except Fenton.

We claim:

1. A method for eliminating or reducing the size of an aggregate of macromolecules in the eye, the method comprising administering a therapeutically effective amount of an ophthalmic formulation comprised of (a) a noncytotoxic chelating agent containing at least three negatively charged chelating atoms, and (b) a charge-masking agent containing at least one polar electrophilic atom and having a molecular weight less than about 250, wherein the molar ratio of the charge-masking agent to the chelating agent is sufficient to ensure that substantially all negatively charged chelating atoms are associated with a polar electrophilic atom on the charge-masking agent.

2. The method of claim 1, wherein the noncytotoxic chelating agent is a basic addition salt of a polyacid.

3. The method of claim 2, wherein the polyacid is selected from polycarboxylic acids, polysulfonic acids, and polyphosphonic acids.

4. The method of claim 3, wherein the polyacid is a polycarboxylic acid.

5. The method of claim 4, wherein the basic addition salt is a metal salt.

6. The method of claim 1, wherein the charge-masking agent contains two polar electrophilic atoms.

7. The method of claim 1, wherein the at least one polar electrophilic atom in the charge-masking agent is an oxygen atom.

8. The method of claim 6, wherein the two polar electrophilic atoms are oxygen atoms.

9. The method of claim 8, wherein the charge-masking agent has the structure of formula (I) (I) 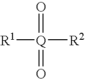
wherein R¹ and R² are independently selected from C₁-C₆ alkyl, C₁-C₆ heteroalkyl, C₆-C₁₄ aralkyl, and C₂-C₁₂ heteroaralkyl, and Q is S or P.

10. The method of claim 9, wherein R¹ and R² are independently selected from C₁-C₃ alkyl, C₁-C₃ heteroalkyl, C₆-C₈ aralkyl, and C₄-C₁₀ heteroaralkyl, and Q is S.

11. The method of claim 10, wherein R¹ and R² are C₁-C₃ alkyl.

12. The method of claim 11, wherein R¹ and R² are methyl.

13. The method of claim 1, wherein the chelating agent is a basic addition salt of a tetracarboxylic acid, the charge-masking agent has the structure of formula (I) 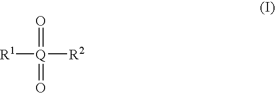
wherein R¹ and R² are independently selected from C₁-C₆ alkyl, C₁-C₆ heteroalkyl, C₆-C₁₄ aralkyl, and C₂-C₁₂ heteroaralkyl, and Q is S or P, and the molar ratio of the charge-masking agent to the chelating agent is in the range of 2:1 to 12:1.

14. The method of claim 13, wherein the molar ratio of the charge-masking agent to the chelating agent is in the range of 4:1 to 10:1.

15. The method of claim 14, wherein the molar ratio of the charge-masking agent to the chelating agent is about 8:1.

16. The method of claim 1, wherein the charge-masking agent has a molecular weight of less than about 125.

17. The method of claim 1, wherein the formulation further comprises a pharmaceutically acceptable vehicle.

18. The method of claim 17, wherein the vehicle is aqueous.

19. The method of claim 17, wherein the formulation is administered in the form of eye drops.

20. The method of claim 18, wherein the formulation consists essentially of the chelating agent, the charge-masking agent, and the aqueous vehicle.

21. The method of claim 1, wherein the aggregate of macromolecules comprises Advanced Glycation Endproducts.

22. The method of claim 1, wherein the macromolecules are peptidyl compounds.

23. The method of claim 22, wherein the macromolecules are proteins.

24. The method of claim 22, wherein the macromolecules are lipoproteins.

25. The method of claim 1, wherein the macromolecules are lipids.

26. The method of claim 1, wherein the macromolecules are polynucleotides.

27. The method of claim 1, wherein the formulation further includes an ophthalmologically active agent.

28. The method of claim 1, wherein the chelating agent represents at least 0.6 wt. % of the formulation.

29. A formulation comprising:
(a) a noncytotoxic chelating agent containing at least three negatively charged chelating atoms;
(b) a charge-masking agent containing at least one polar group and having a molecular weight less than about 250, wherein the polar group contains at least one heteroatom having a Pauling electronegativity of greater than about 3.00, and further wherein the molar ratio of the charge-masking agent to the chelating agent is sufficient to ensure that substantially all negatively charged chelating atoms are associated with a heteroatom on the charge-masking agent; and
(c) a pharmaceutically acceptable vehicle.

30. The formulation of claim 29, wherein the noncytotoxic chelating agent is a basic addition salt of a polyacid.

31. The formulation of claim 30, wherein the polyacid is selected from polycarboxylic acids, polysulfonic acids, and polyphosphonic acids.

32. The formulation of claim 31, wherein the polyacid is a polycarboxylic acid.

33. The formulation of claim 32, wherein the basic addition salt is a metal salt.

34. The formulation of claim 29, wherein the polar group of the charge-masking agent contains two heteroatoms.

35. The formulation of claim 29, wherein the at least one heteroatom in the charge-masking agent are oxygen atoms.

36. The formulation of claim 34, wherein the two heteroatoms are oxygen atoms.

37. The formulation of claim 36, wherein the charge-masking agent has the structure of formula (I) 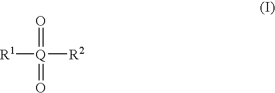
wherein R¹ and R² are independently selected from C₁-C₆ alkyl, C₁-C₆ heteroalkyl, C₆-C₁₄ aralkyl, and C₂-C₁₂ heteroaralkyl, and Q is S or P.

38. The formulation of claim 37, wherein R¹ and R² are independently selected from C₁-C₃ alkyl, C₁-C₃ heteroalkyl, C₆-C₈ aralkyl, and C₄-C₁₀ heteroaralkyl, and Q is S.

39. The formulation of claim 38, wherein R¹ and R² are C₁-C₃ alkyl.

40. The formulation of claim 39, wherein R¹ and R² are methyl.

41. The formulation of claim 29, wherein the chelating agent is a basic addition salt of a tetracarboxylic acid, the charge-masking agent has the structure of formula (I) 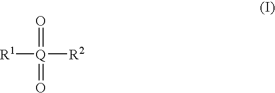
wherein R¹ and R² are independently selected from C₁-C₃ alkyl, C₁-C₃ heteroalkyl, C₆-C₈ aralkyl, and C₄-C₁₀ heteroaralkyl, and Q is S or P, and the molar ratio of the charge-masking agent to the chelating agent is in the range of 2:1 to 12:1.

42. The formulation of claim 41, wherein the molar ratio of the charge-masking agent to the chelating agent is in the range of 4:1 to 10:1.

42. The formulation of claim 42, wherein the molar ratio of the charge-masking agent to the chelating agent is about 8:1.

43. The formulation of claim 29, wherein the charge-masking agent has a molecular weight of less than about 125.

44. The formulation of claim 29, wherein the vehicle is an aqueous vehicle.

45. The formulation of claim 29, consisting essentially of the chelating agent, the charge-masking agent, and the aqueous vehicle.

46. A sterile ocular insert for delivery of an ophthalmic formulation to the eye, comprising a controlled release implant housing the formulation of claim 29 and suitable for implantation into the conjunctiva, sclera, pars plana, anterior segment or the posterior segment of the eye.

47. The ocular insert of claim 46, wherein the implant is comprised of a polymeric matrix that gradually releases the formulation to the eye through diffusion and/or matrix degradation.

48. The ocular insert of claim 47, wherein the polymeric matrix is completely biodegradable.

49. The ocular insert of claim 46, wherein the implant is comprised of a laminated structure in which an inner core housing the formulation is contained between outer layers of a permeable polymer through which the formulation gradually diffuses.

50. A method for preventing or treating a mammalian individual susceptible to or afflicted with an adverse ocular condition, comprising topically administering the formulation of claim 29 to an eye of the individual.

51. The method of claim 50, wherein the adverse ocular condition is associated with oxidative and/or free radical damage to the eye.

52. The method of claim 50, wherein the adverse ocular condition is a condition, disease, or disorder of the cornea, retina, lens, sclera, anterior segment, or posterior segment of the eye.

53. The method of claim 50, wherein the adverse ocular condition is associated with aging.

54. The method of claim 50, wherein the adverse ocular condition is secondary cataract formation.
